(19) United States
(12) Patent Application Publication
(10) Pub. No.: US 2023/0285383 A1
(43) Pub. Date: Sep. 14, 2023

(54) TREATMENT OF SKIN DISEASES

(71) Applicant: Ichilov Tech Ltd., Tel-Aviv (IL)

(72) Inventors: Eli SPRECHER, Givatayim (IL); Ofer SARIG, Ramat-Yishai (IL)

(73) Assignee: Ichilov Tech Ltd., Tel-Aviv (IL)

(21) Appl. No.: 18/130,943

(22) Filed: Apr. 5, 2023

Related U.S. Application Data
(63) Continuation of application No. PCT/IL2021/051197, filed on Oct. 5, 2021.
(60) Provisional application No. 63/087,353, filed on Oct. 5, 2020.

(51) Int. Cl. A61K 31/4745; A61P 17/06; A61P 29/00; A61K 9/00

(52) U.S. Cl. CPC A61K 31/4745; A61P 17/06; A61P 29/00; A61K 9/0014

(57) ABSTRACT

Compounds represented by Formula A as defined in the specification for use in treating skin diseases such as, for example, psoriasis, or atopic dermatitis, in a subject in need thereof are disclosed. Compositions containing these compounds, formulated for topical application, are also disclosed.

### RELATED APPLICATIONS

This application is a Continuation of PCT Patent Application No. PCT/IL2021/051197 having International filing date of Oct. 5, 2021, which claims the benefit of priority under 35 USC § 119(e) of U.S. Provisional Patent Application No. 63/087,353 filed on Oct. 5, 2020. The contents of the above applications are all incorporated by reference as if fully set forth herein in their entirety.

### SEQUENCE LISTING STATEMENT

The XML file, entitled 96178SequenceListing.xml, created on Apr. 4, 2023, comprising 5,907 bytes, submitted concurrently with the filing of this application is incorporated herein by reference.

### FIELD AND BACKGROUND OF THE INVENTION

The present invention, in some embodiments thereof, relates to therapy and, more particularly, but not exclusively, to novel methods and compositions for the treatment of skin diseases.

Familial tumoral calcinosis (FTC) represents a clinically and genetically heterogeneous group of inherited diseases manifesting with dermal and subcutaneous deposition of calcified materials. It was previously demonstrated that the normophosphatemic variant of FTC (NFTC) is caused by mutations in the sterile alpha motif domain 9 (SAMD9) gene which encodes a 170 kD protein [Chefetz, I. et al. (2008) J Invest Dermatol 128: 1423-9]. NFTC is inherited in an autosomal recessive manner, and has been exclusively reported in Yemenite Jews. Several inflammatory cytokines, including tumor necrosis factor alpha (TNF-α) and interferon-gamma (IFN-γ), regulate SAMD9 gene expression [Chefetz, I. et al. (2008), supra], which may explain the fact that in NFTC, inflammation seems to precede ectopic calcification in the skin.

It was previously established that SAMD9 may function by inhibiting EGR1 (Early growth response protein 1) expression [Hershkovitz, D. et al. (2011) J Invest Dermatol 131: 662-9]. The EGR1 gene product is a transcription factor with roles in differentiation and growth. EGR1 is also an important mediator of inflammation and may be involved in the pathogenesis of Crohn's disease, where SAMD9 is down-regulated, and scleroderma, a disorder notoriously featuring ectopic calcification. Moreover, tissue deposition of calcium phosphate has been associated with increased EGR1 expression [Molloy, E. S. and McCarthy, G. M. (2006) Curr Opin Rheumatol 18: 187-92]. EGR1 has also been implicated in the pathogenesis of breast, prostate, and lung cancer and may be important for metastatic progression due to the activation of genes that control actin contractility [Cermak, V. et al. (2010) Cell Mol Life Sci 67: 3557-68], an observation that is in line with data showing intracellular redistribution of actin filaments following downregulation of SAMD9 [Hershkovitz, D. et al. (2011), supra].

WO 2016/174674 describes studies conducted for uncovering molecules that are inducers of SAMD9 transcriptional activity, which down-regulate EGR1 and as such can be used for the treatment of inflammatory and hyperproliferative diseases including skin diseases, such as psoriasis.

Camptothecin (CPT) and structural analogs (derivatives) thereof have been described in the art as anti-cancer agent. See, for example, Fengzhi et al., Am. J. Cancer Res. 2017; 7(12): 2350-2394.

U.S. Patent Application having Publication Nos. 2008/0107720, 2004/0223971, 2004/0010001, 2009/0214474 and 2015/0056192, KR 2011/010609, CN 109553608A, and WO 2014/179528 all teach combination therapies in which cytotoxic agents such as camptothecin (CPT) and structural analogs (derivatives) thereof are combined with additional agents for treating cancer and/or other proliferative or inflammatory conditions.

Additional background art includes U.S. Patent Application having Publication No. 2014/0011812, WO 2014/011540 and EP Patent No. 0502668.

### SUMMARY OF THE INVENTION

According to an aspect of some embodiments of the present invention there are provided compounds of the camptothecin family, as defined herein, for use in treating a skin disease in a subject in need thereof.

According to an aspect of some embodiments of the present invention there are provided compounds of the camptothecin family, as defined herein, for use in treating inflammation and/or an autoimmune disease and/or hyperfroliferative disease or disorder in a subject in need thereof.

According to an aspect of some embodiments of the present invention there is provided a compound represented by Formula A:

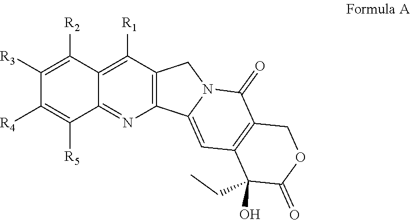

or a pharmaceutically acceptable salt thereof, or a carboxylate form thereof,
wherein:
R₁-R₅ are each independently selected from hydrogen, alkyl, alkenyl, allyl, cycloalkyl, halo, trihaloalkyl, amino, alkoxy, thioalkoxy, hydroxyl, thiol, nitro, cyano, aryl, heteroaryl, silyl, oxime, carboxylate, thiocarboxylate, carbamate, thiocarbamate, or, alternatively or in addition, two of R₁-R₅ form together a cyclic ring, the cyclic ring being selected from aryl, heteroaryl, cycloalkyl or heteroalicyclic, each being independently substituted or unsubstituted,
for use in treating a skin disease in a subject in need thereof.

According to some of any of the embodiments described herein, each of R₁-R₅ is hydrogen.

According to some of any of the embodiments described herein, R₃ is hydroxy.

According to some of any of the embodiments described herein, R₁ is a substituted or unsubstituted alkyl.

According to some of any of the embodiments described herein, R₃ is a carboxylate.

According to some of any of the embodiments described herein, the compound is selected from camptothecin, irinotecan, topotecan, and SN-38.

According to some of any of the embodiments described herein, the compound is selected from Camptothecin, Irinotecan, Topotecan, Rubitecan, Belotecan, Exatecan, Lurtotecan, Diflomotecan, Gimatecan, Karenitecin, Silatecan, Namitecan, Elomotecan, DRF-1042, MAG-CPT, BAY 38-3441, Delimotecan, Chimmitecan and Simmitecan.

According to some of any of the embodiments described herein, the compound is capable of inducing SAMD9 expression and/or of downregulating EGR1.

According to some of any of the embodiments described herein, the skin disease is an inflammatory skin disease.

According to some of any of the embodiments described herein, the disease is a chronic inflammatory disease.

According to some of any of the embodiments described herein, the disease is an acute inflammatory disease.

According to some of any of the embodiments described herein, the skin disease is a hyperproliferative skin disease.

According to some of any of the embodiments described herein, the skin disease is selected from the group consisting of inflammation, an infectious disease, an autoimmune disease, a hypersensitivity associated inflammation, a graft rejection and an injury.

According to some of any of the embodiments described herein, the skin disease is selected from the group consisting of an atopic dermatitis, a contact dermatitis, a dermatitis herpetiformis, a generalized exfoliative dermatitis, a seborrheic dermatitis, a psoriasis, a drug rash, an erythema multiforme, an erythema nodosum, a granuloma annulare, a poison ivy, a poison oak, a toxic epidermal necrolysis, an acne and a rosacea.

According to some of any of the embodiments described herein, the skin disease is psoriasis.

According to some of any of the embodiments described herein, the skin disease is atopic dermatitis.

According to some of any of the embodiments described herein, the treating comprises topical application of the compound onto a skin of the subject.

According to an aspect of some embodiments of the present invention there is provided a pharmaceutical composition comprising a CPT compound, or a compound represented by Formula A, as defined and/or described herein in any of the respective embodiments and any combination thereof, and a pharmaceutically acceptable carrier, the composition being formulated for topical application onto a skin of a subject in need thereof.

According to some of any of the embodiments described herein, the composition is for use in treating a skin disease in the subject.

According to some of any of the embodiments described herein, the composition is configured or formulated such that the compound is locally present in the epidermis and/or upper dermis.

According to some of any of the embodiments described herein, the composition is configured or formulated such that a presence of the compound in the physiological system of the subject is minimized or null.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B presents bar graphs showing induction of SAMD9 expression in different Hella cells treated with CPT. FIG. 1A is a bar graph SAMD9 expression in HeLa Cells, TERC-transformed fibroblasts and primary fibroblasts treated with CPT. All three cell types were cultured in 12-well plates and treated with 5 μM of CPT for 24 hours in duplicates. FIG. 1B is a bar graph showing SAMD9 expression in primary fibroblasts in the presence of DMSO or 1 μM, 2 μM, 5 μM and 10 μM CPT. SAMD9 expression was measured via qRT-PCR, in triplicates. Results are expressed as fold-change in SAMD9 RNA expression relative to control cells treated with DMSO±standard error. *=p<0.05; **=p<0.01.

FIG. 2 is a bar graph showing the induction of SAMD9 expression and repression of EGR1 in Hela cells treated with CPT. Cells were cultured in duplicates in 12-well plates in the presence of DMSO or 10 μM of CPT for 24, 48 and 72 hours. SAMD9 and EGR1 expression were measured via qRT-PCR, all samples were run in triplicates. Results are expressed as SAMD9 or EGR1 RNA expression relative to control cells treated with DMSO±standard error. *=p<0.05; **=p<0.01.

FIGS. 3A-3B are bar graphs showing induction of SAMD9 expression and repression of EGR1 in Hela cells treated with CPT-11 (irinotecan). Cells were cultured in duplicates in 12-well plates in the presence of DMSO or 1 μM, 2 μM, 5 μM and 10 μM CPT-11 for 48 hours (FIG. 3A), or with 10 μM of CPT-11, or DMSO for 48 and 72 hours (FIG. 3B). SAMD9 and EGR1 expression were measured via qRT-PCR, all samples were run in triplicates. Results are expressed as SAMD9 or EGR1 RNA expression relative to control cells treated with DMSO±standard error. *=p<0.05; **=p<0.01, ***=p<0.005.

FIGS. 4A-4D are photographs of a histopathological analysis showing the effect of CPT on imiquimod-induced psoriasiform dermatitis in mice. Two groups of mice were treated 5 times weekly topically with imiquimod and then received for five days either i.p. injection of vehicle only (1% DMSO in 20% lipofuscin) (FIGS. 4A and 4C); or i.p. injection of 7.5 mg/kg CPT in the vehicle (FIGS. 4B and 4D). Biopsies were obtained on day 6 and stained with H&E (upper panels) and Ki67 (lower panels).

FIG. 5 is a bar graph showing Ki67 staining and epidermal thickness after treatment with CPT of imiquimod-induced psoriasiform dermatitis. Eight Balb/c mice were treated with imiquimod 5% topically daily for 5 days and then divided into two equal groups which received c for 5 days i.p. injection of vehicle only (1% DMSO in 20% lipofuscin) or 7.5 mg/kg/day CPT in the vehicle. The epidermal thickness (black columns) and the positive Ki67 in the epidermis (white columns) are presented in percentage relative to control mice treated with the vehicle treated group±standard error. *=p<0.05.

FIG. 6 is a bar graph showing the overall effect of CPT and CPT-11 in chimeric mice carrying human psoriatic skin. Six weeks after human skin grafting, chimeric mice were treated as follows: one group of mice was injected i.p. five times a week with the vehicle (1% DMSO and 5% ethanol in 20% lipofuscin); a second group of mice was injected five times a week CPT (3 mg/kg); a third group of mice was injected 3 times a week CPT-11 (50 mg/kg); a fourth group of mice was injected 3 times a week CPT-11 (30 mg/kg); and a fifth group of mice, was treated with dexamethasone (DEX) cream applied 5 times a week on the graft, as a positive control anti-inflammatory agent. Each group included six mice, and the treatment was performed for a total of 10 days. The grafts were harvested from the four groups of mice, paraffin-embedded, stained for hematoxylin and eosin (H&E), analyzed, and scored for the overall improvement of mice in teach group.

FIGS. 7A-7H are photographs of the histopathological analysis showing the effect of CPT and CPT-11 in chimeric mice carrying human psoriatic skin. FIGS. 7A and 7E show data obtained for mice treated by i.p. injection five times a week of the vehicle only (1% DMSO and 5% ethanol in 20% lipofuscin). FIGS. 7B and 7F show data obtained for mice treated with i.p. injection five times a week of CPT (3 mg/kg). FIGS. 7C and 7G) present data obtained for mice treated by I.P injection three times a week of CPT-11 (30 mg/kg). FIGS. 7D and 7H present data obtained for mice treated by I.P injection three times a week of CPT-11 (50 mg/kg). Each group included six mice, and the treatment was performed for a total of 10 days. Biopsies were obtained on day 10 and stained with H&E (upper panels) and Ki67 (lower panels).

FIG. 8 is a bar graph presenting data of the Ki67 staining and epidermal thickness in chimeric mice carrying human psoriatic skin after treatment with CPT and CPT-11. Mice were treated as described in FIGS. 7A-H, above. Epidermal thickness was measured in micrometers. The epidermal thickness (black columns) and the positive Ki67 in the epidermis (white columns) are presented in percentage relative to control mice treated with the vehicle treated group±standard error. *=p<0.05. Results represent the average Epidermal thickness and Ki67 staining for each mice group±SE (*p<0.05; ** p<0.001, *** p<0.0005).

FIG. 9 is a bar graph presenting the inflammation index in chimeric mice carrying human psoriatic skin after treatment with CPT and CPT-11, as described in FIGS. 7A-H. Results represent the average inflammation index for each mice group±SE (** p<0.001, *** p<0.0005).

FIG. 10A presents RNA-seq analysis of HeLa cells treated with CPT. HeLa cells were treated with 10 μM CPT or DMSO for 48 hours (three independent experiments). Total RNA was extracted and sent to RNA-seq. Volcano plot represents the number of genes that were differentially expressed. Red points mark the genes that were significantly upregulated and blue points mark the genes that were downregulated (FDR<0.01). The x-axis shows log 2fold-changes in expression and the y-axis the −log of p-value for gene being differentially expressed. P-value for enrichment for genes belongs to a known psoriasis related pathogenic pathways (taken from IPA database) was calculated using hypergeometric distribution (using a background of 24190 genes).

FIG. 10B presents a table summarizing the number of genes in each group, the number of differentially expressed genes, the overlap, and the adjusted p-value (after correcting for multiple testing).

FIG. 11 (Background Art) presents the chemical structures of exemplary compounds of Formula A which are usable in the context of the present embodiments as taken from Fengzhi et al., Am. J. Cancer Res. 2017; 7(12): 2350-2394).

### DESCRIPTION OF SPECIFIC EMBODIMENTS OF THE INVENTION

The present invention, in some embodiments thereof, relates to therapy and, more particularly, but not exclusively, to novel methods and compositions for the treatment of skin diseases.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Conventional treatments for inflammation do not fundamentally cure inflammation, and are often endowed with side effects such as hypersensitivity reaction, and deterioration of immune system.

Psoriasis, a chronic inflammatory disease manifested mainly in skin tissues, affects about 2-3% of the world population. To date there is no cure for psoriasis.

Some of the present inventors have previously uncovered that loss of expression or function of SAMD9 leads to inflammation and subsequent calcinosis. SAMD9 functions by down-regulating the expression of EGR1, a critical regulator of inflammatory responses.

WO 2016/174674, by some of the present inventors, describes studies conducted for uncovering, through laborious experimentation and screening, molecules which act as inducers of SAMD9 transcriptional activity and which thereby down-regulate EGR1. Some of the uncovered molecules were indeed proved to be effective in the treatment of inflammatory and hyperproliferative diseases, including skin diseases such as psoriasis and atopic dermatitis.

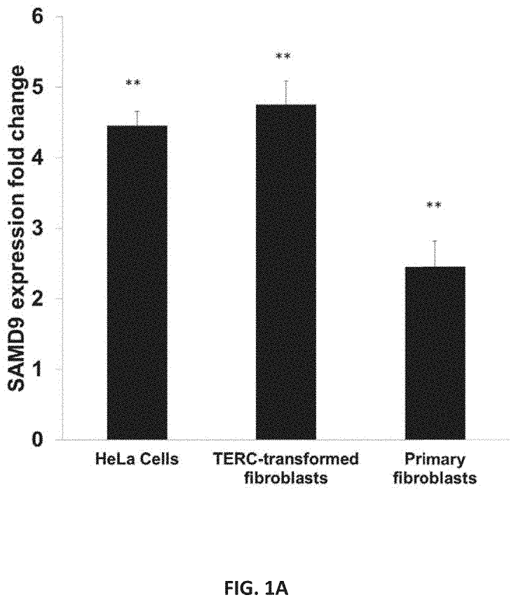

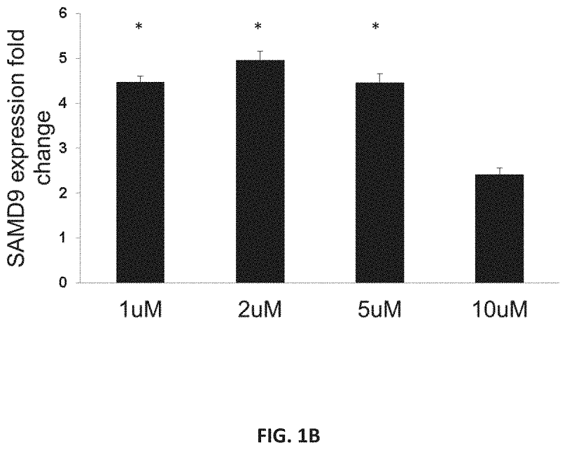

The present inventors have now uncovered that compounds of the camptothecin family (also referred to herein as CPT compounds), which have been described in the art as cytotoxic agents, act as inducers of SAMD9 transcriptional activity and as downregulators of EGR1 (see, for example, FIGS. 1A, 1B, 2, 3A and 3B), and as such are usable in the treatment of inflammatory and hyperproliferative diseases such as described in WO 2016/174674, which is incorporated by reference as if fully set forth herein, and particularly in the treatment of skin diseases (e.g., skin inflammation and/or hyperproliferation). Without being bound to any particular theory, it is assumed that since SAMD9 deficiency was found to manifest exclusively in skin with inflammation and calcinosis (Topaz et al, Am J Hum Genet, 2006), up-regulation of SAMD9 efficiently affects inflammatory skin conditions.

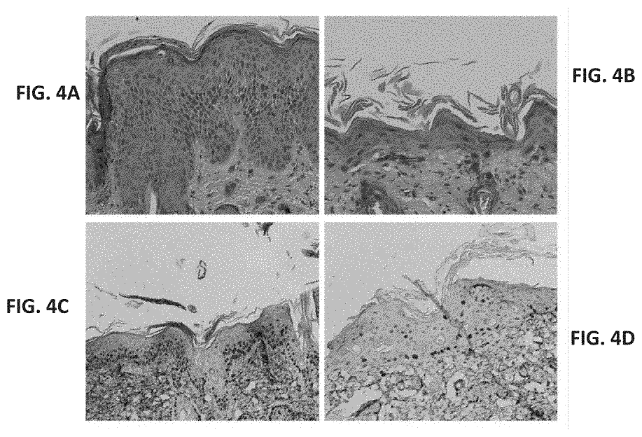

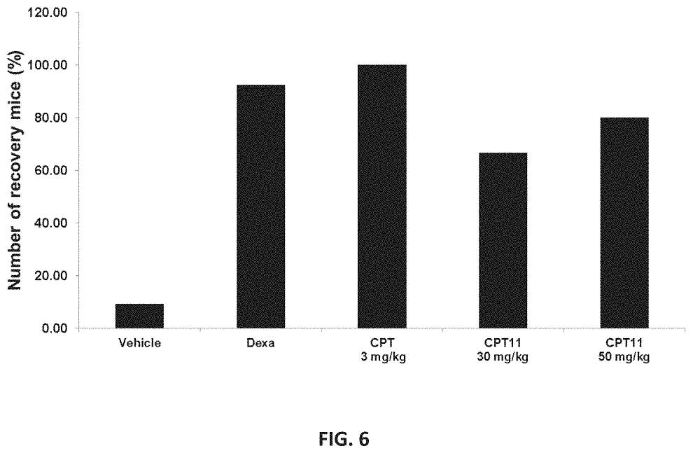

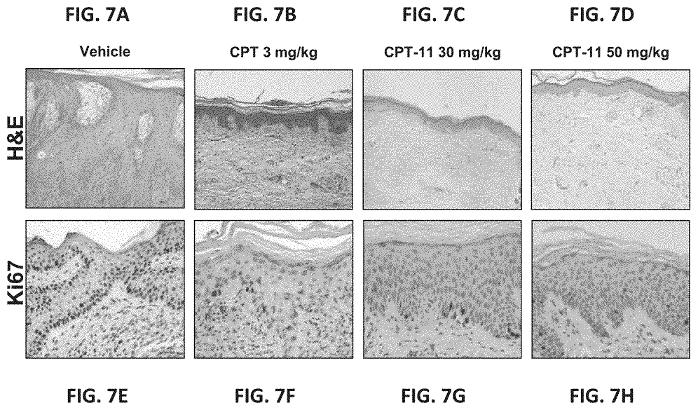

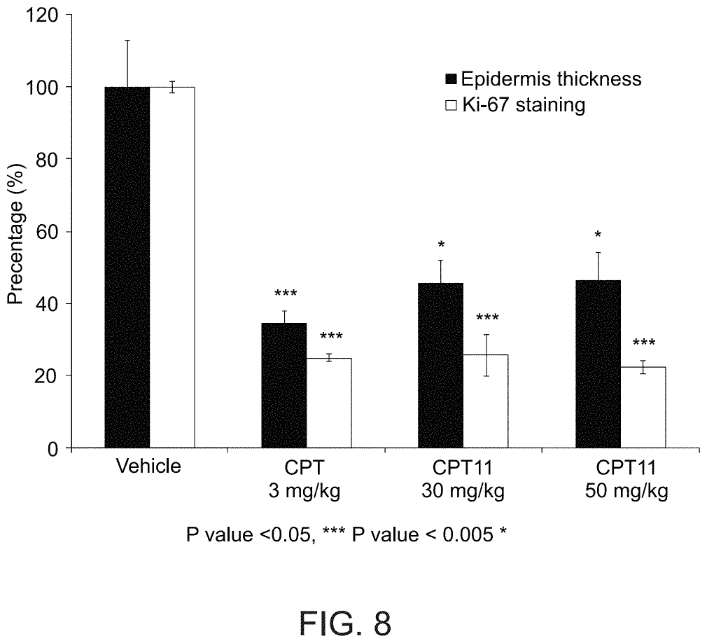

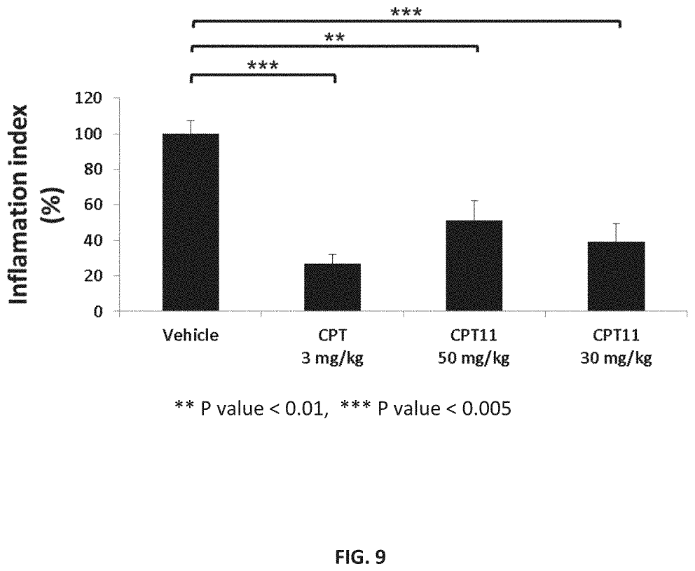

The present inventors have demonstrated that CPT compounds are effective in treating psoriasis, when tested in both mice having imiquimod-induced psoriasiform dermatitis (see, FIGS. 4A-D and 5) and chimeric mice carrying human psoriatic skin (see, FIGS. 6, 7A-H, 8 and 9).

These results indicate that compounds of the camptothecin (CPT) family are suitable for incorporation into pharmaceutical compositions that are formulated for application onto a skin of a subject (e.g., for topical application) and are usable in treating skin diseases, for example, inflammatory and/or hyperproliferative skin diseases such as psoriasis.

Embodiments of the present invention therefore relate to use of camptothecin (CPT) and structural analogs thereof (derivatives thereof), which are collectively represented herein by Formula A, and are also referred to herein as CPT compounds, in the treatment of skin conditions, for example, inflammatory and/or hyperproliferative skin diseases.

Embodiments of the present invention further relate to pharmaceutical compositions comprising camptothecin (CPT) and structural analogs thereof (derivatives thereof), which are collectively represented herein by Formula A, and are also referred to herein as CPT compounds, which are formulated for application to the skin (e.g., for topical application).

Compounds:

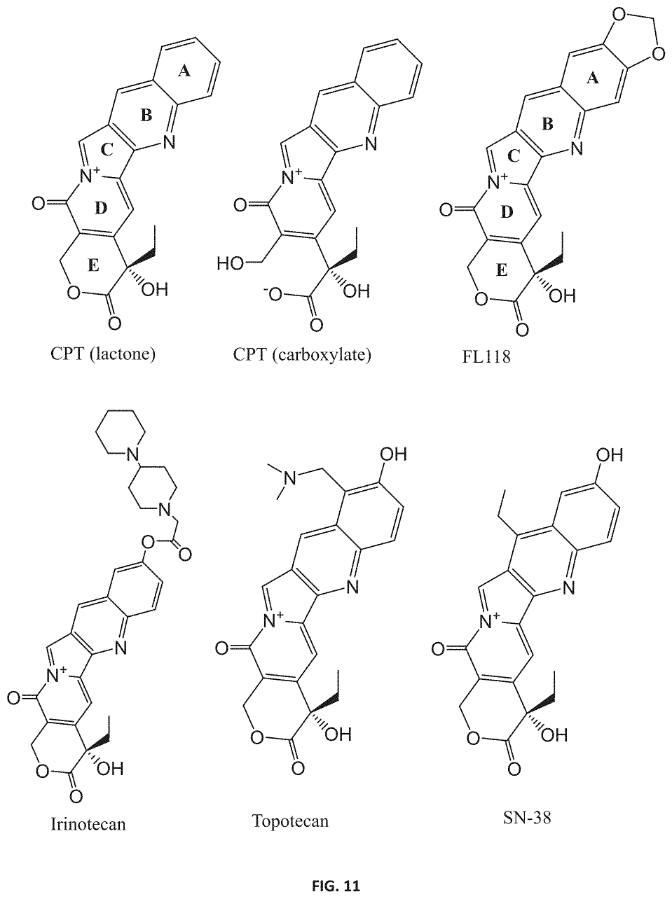

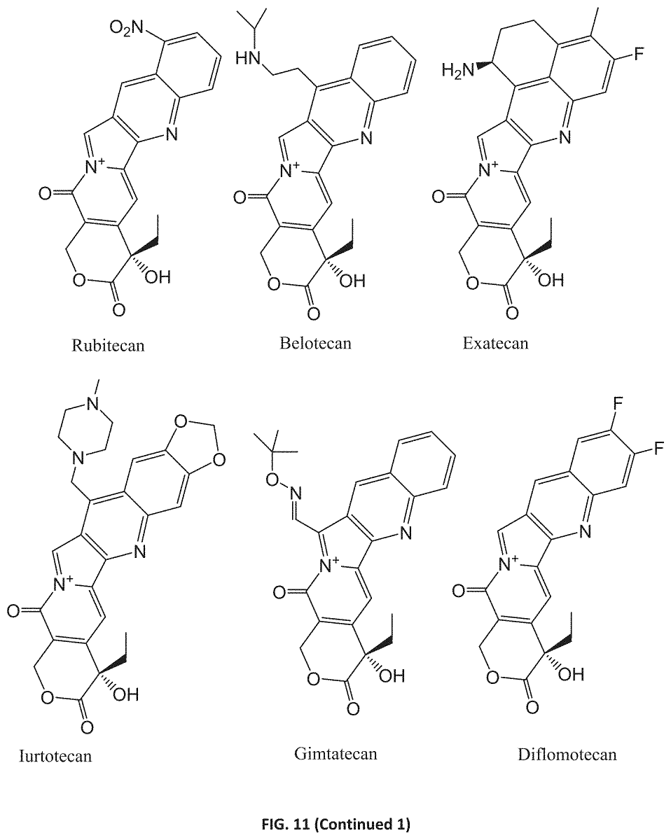

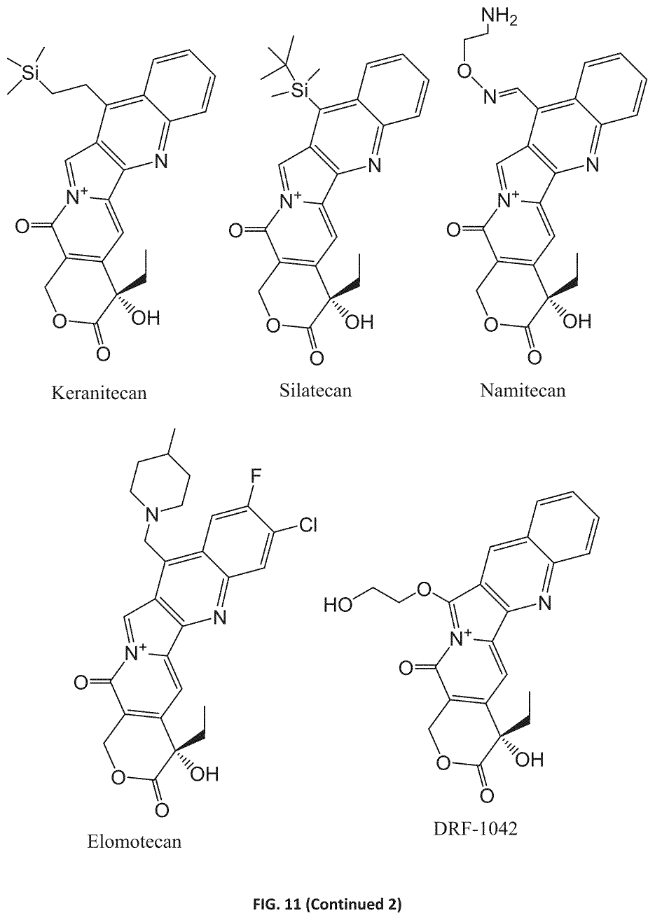

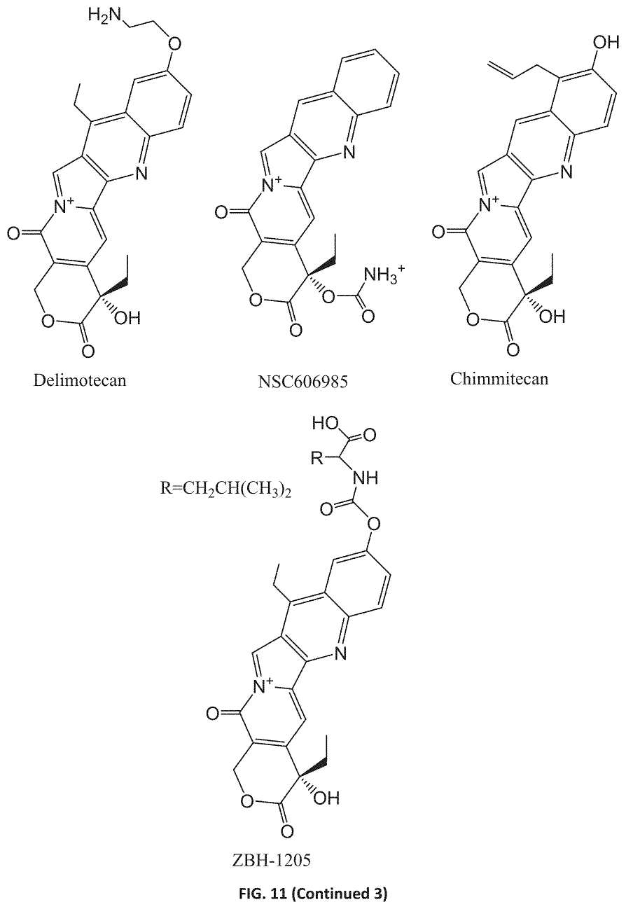

Compound usable in the context of the present embodiments encompass camptothecin (CPT) and structural analogs thereof (derivatives thereof). Any structural analog of CPT is contemplated. Exemplary compounds are presented in FIG. 11.

According to some of any of the embodiments described herein, the compounds can be collectively represented by Formula A:

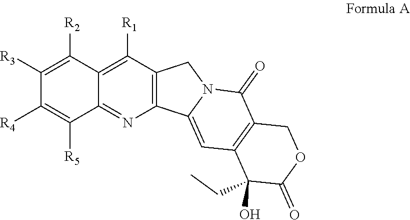

Compounds of Formula A feature a polycyclic skeleton made of 4 fused, substantially planar rings, and a fifth ring which is a lactone form of a corresponding carboxylate. The substituents of the lactone are a hydroxy and ethyl, and feature a stereoconfiguration as indicated in Formula A.

The polycyclic skeleton can be decorated by 1, 2, 3, 4 or 5 substituents (other than hydrogens), denoted as R₁-R₅ in Formula A.

When R₁-R₅ are each hydrogen, the compound is camptothecin (CPT).

Alternatively, one or more of R₁-R₅ is other than hydrogen and can independently be, for example, alkyl, alkenyl, allyl, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine, amine-oxide, oxo, oxime, thiohydrazine, hydrazide, thiohydrazide, silyl or hydrazine.

In some embodiments, one or more of R₁-R₅ is other than hydrogen and can independently be, for example, alkyl, alkenyl, allyl, cycloalkyl, halo, trihaloalkyl, amino, alkoxy, thioalkoxy, hydroxyl, thiol, nitro, cyano, aryl, heteroaryl, silyl, oxime, carboxylate, thiocarboxylate, carbamate, or thiocarbamate.

Additionally, or alternatively, two of R₁-R₅ form together a cyclic ring, which can be an aryl, a heteroaryl, a cycloalkyl or a heteroalicyclic, as these terms are defined herein, and each can independently be substituted or unsubstituted.

In some of any of the embodiments described herein, R₃ is hydroxy, thiohydroxy, alkoxy or thioalkoxy. In some embodiments, R₃ is hydroxy. Exemplary such CPT compounds include, but are not limited to, topotecan and SN-38 (see, FIG. 11).

In some of any of the embodiments described herein, R₁ is a substituted or unsubstituted alkyl, preferably a short (C1-6 or C1-4) alkyl, such as methyl, ethyl, propyl, butyl, pentyl or hexyl.

In some of any of the embodiments described herein, R₁ is an unsubstituted alkyl, preferably a short (C1-6 or C1-4) unsubstituted alkyl, such as methyl, ethyl, propyl, butyl, pentyl or hexyl.

In some of any of the embodiments described herein, R₁ is a substituted or unsubstituted ethyl.

In some of any of the embodiments described herein, R₁ is an unsubstituted ethyl. Exemplary such CPT compounds include, for example, irinotecan and SN-38 (see FIG. 11).

In some of any of the embodiments described herein, R₁ is a substituted or unsubstituted alkyl, as described herein in any of the respective embodiments, and R₃ is hydroxy. An exemplary such compound is SN-38 (see FIG. 11).

In some of any of the embodiments described herein, R₃ is a carboxylate, and in some embodiments, it is an O-carboxylate, as defined herein.

In some of these embodiments, the carboxylate is a —O—C(═O)—R′ group and R′ is an alkyl, which can be substituted or unsubstituted.

In some of these embodiments, R′ in the carboxylate is a substituted alkyl, and in some of these embodiments, the alkyl is substituted by a heteroaryl.

In some of any of these embodiments, R′ in the carboxylate is a substituted alkyl, and the alkyl is a lower alkyl (C1-4), and is, for example, a methylene, ethylene, propylene, or butylene, preferably, methylene or ethylene, more preferably methylene.

In some of any of the embodiments described herein, R₃ is O-carboxylate, —O—C(═O)—R′ and R′ is a substituted methylene. In some of these embodiments, the methylene is substituted by a heteroaryl, for example, a substituted piperidine. In some of these embodiments described herein, R₁ is a substituted or unsubstituted alkyl, as described herein in any of the respective embodiments. An exemplary such compound is irinotecan (see FIG. 11).

In some of any of the embodiments described herein, one or more of R₂-R₅ is/are independently a halo.

In some of any of the embodiments described herein, two or more of R₂-R₅ form together a cyclic ring, and in some of these embodiments, the cyclic ring is a heteroalicyclic ring, for example, a dioxane.

In some of any of the embodiments described herein, R₁ and one of R₂-R₅ form together a cyclic ring, and in some of these embodiments, the cyclic ring is an alicyclic ring, which can be substituted or unsubstituted, as described herein.

In some of any of the embodiments described herein, R₁ is a substituted alkyl, preferably a short (C1-6 or C1-4) unsubstituted alkyl, such as methyl, ethyl, propyl, butyl, pentyl or hexyl.

In some of any of the embodiments described herein, R₁ is a substituted methyl or ethyl, and the substituent can be, for example, a heteroalicyclic, a silyl, amine, amine-oxide, oxime, or any of the substituents described herein.

In some of any of the embodiments described herein, R₁ is silyl.

In some of any of the embodiments described herein, R₂ is an electron withdrawing group, for example, nitro.

In some of any of the embodiments described herein, R₃ is an electron donating group, for example, hydroxy or alkoxy or fluoro.

Exemplary CPT compounds of Formula A are presented in FIG. 11.

In some embodiments, the CPT compound is irinotecan.

In some embodiments, the CPT compound is topotecan.

In some embodiments, the CPT compound is SN-38.

In some embodiments, the CPT compound is one or more of Camptothecin, Irinotecan, Topotecan, Rubitecan, Belotecan, Exatecan, Lurtotecan, Diflomotecan, Gimatecan, Karenitecin, Silatecan, Namitecan, Elomotecan, DRF-1042, MAG-CPT, BAY 38-3441, Delimotecan, Chimmitecan and Simmitecan.

According to some embodiments of the present invention, there is provided a compound selected from the compounds presented in FIG. 11, for use in any of the methods, uses and compositions as described herein.

According to some embodiments of the present invention, there is provided a compound selected from Camptothecin, Irinotecan, Topotecan, Rubitecan, Belotecan, Exatecan, Lurtotecan, Diflomotecan, Gimatecan, Karenitecin, Silatecan, Namitecan, Elomotecan, DRF-1042, MAG-CPT, BAY 38-3441, Delimotecan, Chimmitecan and Simmitecan, for use in any of the methods, uses and compositions as described herein.

Embodiments of the present invention also encompass any of the compounds of Formula A as described herein, when in a carboxylate form thereof.

Herein, a carboxylate form means that the lactone ring is in its hydrolyzed form, and includes a free carboxylate or carboxylic acid end group and a free hydroxy group instead of the lactone, as is exemplified, for example, in FIG. 11, for CPF.

For any of the embodiments described herein, the compound may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound.

In the context of some of the present embodiments, a pharmaceutically acceptable salt of the compounds described herein may optionally be a base addition salt comprising at least one acidic (e.g., phenol and/or carboxylic acid) group of the compound which is in a negatively charged form (e.g., wherein the acidic group is deprotonated), in combination with at least one counter-ion, derived from the selected base, that forms a pharmaceutically acceptable salt.

The base addition salts of the compounds described herein may therefore be complexes formed between one or more acidic groups of the drug and one or more equivalents of a base.

The base addition salts may include a variety of organic and inorganic counter-ions and bases, such as, but not limited to, sodium (e.g., by addition of NaOH), potassium (e.g., by addition of KOH), calcium (e.g., by addition of Ca(OH)₂, magnesium (e.g., by addition of Mg(OH)₂), aluminum (e.g., by addition of Al(OH)₃ and ammonium (e.g., by addition of ammonia). Each of these acid addition salts can be either a mono-addition salt or a poly-addition salt, as these terms are defined herein.

In the context of some of the present embodiments, a pharmaceutically acceptable salt of the compounds described herein may optionally be an acid addition salt comprising at least one base group (e.g., amine or amide group) of the compound which is in a positively charged form (e.g., wherein an —NH— group is protonated), in combination with at least one counter-ion, derived from the selected acid, that forms a pharmaceutically acceptable salt.

The acid addition salts of the compounds described herein may therefore be complexes formed between one or more basic groups of the drug and one or more equivalents of an acid.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Depending on the stoichiometric proportions between the charged group(s) in the compound and the counter-ion in the salt, the acid or base additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of the compound is modified by an acyl group, optionally (C₁₋₄)acyl (e.g., acetyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The present embodiments further encompass any isomorph of a compound as described herein, when the compound exhibits polymorphism.

The present embodiments further encompass any enantiomers and diastereomers of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

Methods and Uses:

According to an aspect of some embodiments of the present invention there is provided a method of treating an inflammation or a hyperproliferative disease in a subject in need thereof, the method comprising administering to the subject a therapeutically effective amount of a compound represented by Formula A, as described herein in any of the respective embodiments, thereby treating the inflammation or the hyperproliferative disease in the subject.

According to an aspect of some embodiments of the present invention there is provided a use of a compound represented by Formula A, as described herein in any of the respective embodiments, in the manufacture of a medicament for treating an inflammation or a hyperproliferative disease in a subject in need thereof.

According to an aspect of the present invention, there is provided a compound represented by Formula A, as described herein in any of the respective embodiments, for use in the treatment of inflammation or a hyperproliferative disease in a subject in need thereof.

As used herein, the term “treating” refers to alleviating, attenuating, palliating or eliminating the symptoms of a disease, slowing, reversing or arresting the progression of the disease, or curing the disease, with respect to any of the diseases or conditions as described herein.

As used herein, the term “subject” or “subject in need thereof” refers to a mammal, preferably a human being, male or female, at any age, which suffers from the pathology or is at risk to develop the pathology.

According to one embodiment, the pathology is an inflammation or a hyperproliferative disease.

According to one embodiment, the pathology is a skin disease, as described herein.

According to one embodiment, the pathology is an inflammatory or a hyperproliferative skin disease.

According to an aspect of some embodiments of the present invention there is provided a method of treating a skin disease in a subject in need thereof, the method comprising administering to the subject a therapeutically effective amount of a compound represented by Formula A, as described herein in any of the respective embodiments, thereby treating the skin disease in the subject.

According to an aspect of some embodiments of the present invention there is provided a use of a compound represented by Formula A, as described herein in any of the respective embodiments, in the manufacture of a medicament for treating an a skin disease in a subject in need thereof.

According to an aspect of the present invention, there is provided a compound represented by Formula A, as described herein in any of the respective embodiments, for use in the treatment of a skin disease in a subject in need thereof.

Herein throughout, the phrase “skin disease”, is also referred herein interchangeably as “skin condition”, “skin medical condition”, or as, or as part of a “dermatological condition” or “dermatological medical condition”, as these terms are defined hereinafter.

Inflammation

The term “inflammation” as used herein refers to the general term for local accumulation of fluids, plasma proteins, and white blood cells initiated by physical injury, infection, or a local immune response. Inflammation may be associated with several signs e.g. redness, pain, heat, swelling and/or loss of function. Inflammation is an aspect of many diseases and disorders, including, but not limited to, diseases related to immune disorders, viral and bacterial infection, arthritis, autoimmune diseases, collagen diseases, allergy, asthma, pollinosis, and atopy (as described in further detail below).

Thus, inflammation can be triggered by injury, for example injury to skin, muscle, tendons, or nerves Inflammation can be triggered as part of an immune response, e.g., pathologic autoimmune response Inflammation can also be triggered by infection, where pathogen recognition and tissue damage can initiate an inflammatory response at the site of infection.

Herein, the phrase “inflammatory disease” encompasses any medical condition that is associated with (e.g., triggered by or triggers or manifested by) inflammation.

Inflammation according to the present teachings may be associated with chronic (long term) inflammatory diseases or disorders or acute (short term) inflammatory diseases or disorders or medical conditions.

According to some embodiments, the inflammation is associated with a disease selected from the group consisting of an infectious disease, an autoimmune disease, a hypersensitivity associated inflammation, a graft rejection and an injury.

According to a specific embodiment, the inflammation comprises a skin inflammation.

According to a specific embodiment, the skin inflammatory disease is psoriasis.

Diseases characterized by inflammation of the skin (skin inflammatory diseases) include, but are not limited to, dermatitis, atopic dermatitis (eczema, atopy), contact dermatitis, dermatitis herpetiformis, generalized exfoliative dermatitis, seborrheic dermatitis, drug rashes, erythema multiforme, erythema nodosum, granuloma annulare, poison ivy, poison oak, toxic epidermal necrolysis, rosacea, psoriasis and acne.

Inflammation can also result from physical injury to the skin.

Inflammation may be triggered by various kinds of injuries to muscles, tendons or nerves. Thus, for example, inflammation may be caused by repetitive movement of a part of the body i.e. repetitive strain injury (RSI). Diseases characterized by inflammation triggered by RSI include, but are not limited to, bursitis, carpal tunnel syndrome, Dupuytren's contracture, epicondylitis (e.g. tennis elbow), ganglion (i.e. inflammation in a cyst that has formed in a tendon sheath, usually occurring on the wrist), rotator cuff syndrome, tendinitis (e.g., inflammation of the Achilles tendon), tenosynovitis, and trigger finger (inflammation of the tendon sheaths of fingers or thumb accompanied by tendon swelling).

Many diseases related to infectious diseases include inflammatory responses, where the inflammatory responses are typically part of the innate immune system triggered by the invading pathogen Inflammation can also be triggered by physical (mechanical) injury to cells and tissues resulting from the infection. Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases. According to one embodiment, examples of infections characterized by inflammation include, but are not limited to, encephalitis; meningitis; encephalomyelitis; viral gastroenteritis; viral hepatitis.

Furthermore, many immune disorders include acute or chronic inflammation. For example, arthritis is considered an immune disorder characterized by inflammation of joints, but arthritis is likewise considered an inflammatory disorder characterized by immune attack on joint tissues.

Inflammation according to the present teachings may be associated with a deficient immune response (e.g., HIV, AIDS) or with an overactive immune response (e.g., allergy, autoimmune disorders). Thus, inflammation according to the present teachings may be associated with any of the following:

Inflammatory Diseases Associated with Hypersensitivity:

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. and Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases:

Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140). Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to one embodiment, the autoimmune disease is Crohn's disease, psoriasis, scleroderma or rheumatoid arthritis.

Graft Rejection Diseases:

Examples of diseases associated with transplantation of a graft include, but are not limited to, graft rejection, chronic graft rejection, subacute graft rejection, hyperacute graft rejection, acute graft rejection and graft versus host disease.

Allergic Diseases:

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Hyperproliferative Diseases: The term “hyperproliferative disease” as used herein refers to any condition which involves uncontrolled cell growth, i.e. an abnormally high rate of proliferation of cells by rapid cell division.

According to some embodiments of the present invention, the hyperproliferative disease or disorder does not encompass cancers, neoplastic tissues and pre-malignant diseases.

According to some embodiments of the present invention, the hyperproliferative disease is a non-neoplastic or non-malignant hyperproliferative disease or disorder.

According to some embodiments of the present invention, the hyperproliferative disease is a non-neoplastic or non-malignant hyperproliferative skin disease or disorder, for example, psoriasis.

Skin Diseases:

The term “skin” is meant to include skin of the entire embody including the scalp, the forehead, the head, arms, legs, breast and so forth. The term “skin” is also meant to include various layers of the skin, such as stratum corneum, epidermis and dermis.

The term “skin cells” is meant to encompass cells present in stratum corneum, dermis and epidermis. When considering inflammatory dermatological diseases, such cells may include cells that constitute the inflammation in skin, such as T-cells, macrophages, mast cells, Langerhans cells and neutrophils.

A “skin disease” describes a medical condition that adversely affects skin cells. The medical condition can be a skin or dermatological medical condition or can be a systemic condition that is manifested by diseased skin cells.

“Diseased skin cells” include, for example, inflammation in skin cells, injured skin cells, infectious skin cells, and hyperproliferlating skin cells.

Diseased skin cells can result from, for example, an injury, as described herein, an infection, as described herein, an autoimmune disease which is cutaneous, as described herein, or which is manifested by diseased skin cells, a disease that is manifested by hypersensitivity reaction in the skin (e.g., allergy as described herein), inflammation, which can result from a systemic condition and is manifested by skin inflammation or directly from skin inflammation, or from a hyperproliferative skin disease.

Exemplary skin diseases include, but are not limited to, inflammatory dermatological or skin diseases, including any of the inflammatory and/or cutaneous diseases described hereinabove that involve diseased skin cells.

Exemplary skin diseases include, but are not limited to, acne vulgaris, adult eczema, alopecia, allergic contact dermatitis, allergic dermatitis, allergic contact eczema, asteatotic eczema, atopic eczema, hand eczema, atopic dermatitis, childhood eczema, chronic dermatitis of hands or feet, contact dermatitis, contact eczema, discoid eczema, insect bite inflammation, drug-induced skin reactions, dermatitis herpetiformis, discoid lupus erythematosus, eczema, epidermolysis bullosa, erythroderma, erythema nodosum, erythema multiforme, hand eczema, hand and foot dermatitis, ichthyosis vulgaris, infantile eczema, keratoconus, keratosis pilaris lichen simplex chronicus, inflammatory papulosquamous diseases (e.g., lichen planus), nummular dermatitis, over-treatment dermatitis, pemphigus (e.g., pemphigus foliaceus, pemphigus vulgaris), pemphigoid (e.g., bullous pemphigoid), photodermatoses, pityriasis rosea, pyoderma gangrenosum, pompholyx, psoriasis, prurigo nodularis, rosacea, scabies, seborrheic dermatitis, seborrhea, scleroderma, Sjogren's Disease, stasis dermatitis, cutaneous lupus erythematosus (acute, subacute or chronic), sunburn, cutaneous manifestations of systemic lupus erythematosus, vitiligo, vascular diseases (e.g., vasculitis) and urticaria.

Determining if a subject is afflicted with a skin disease as described herein can be performed using a combination of clinical examination, skin biopsy, serological assays, laboratory assays and molecular assays, as is well known in the art.

Effect on SAMD9 and EGR1 Activity:

According to some embodiments of the invention, and without being bound by theory, the compounds as described herein are effective in activating SAMD9 to thereby downregulate EGR1 activity.

As used herein, the term SAMD9 refers to the sterile alpha motif domain containing 9, e.g., human SAMD9, e.g., as set forth in GenBank accession nos. NM_017654.3 or NM_001193307.1 and NP_060124.2 or NP_001180236.1 (mRNA and protein, respectively).

Thus, according to one embodiment, the compounds of the present embodiments upregulate the activity or expression of SAMD9 by about 10%, 20%, 30%, 40%, 50%, 60%, 70%, 80%, 90%, 100% or more, as compared to the activity or expression of the SAMD9 in a cell of the subject prior to the treatment (or in a corresponding sample of another subject having the same pathology and preferably matched with the same species e.g. human, age, weight, sex etc. as the subject in need thereof).

As used herein, the term EGR1 refers to the Early Growth Response protein 1 such as the human EGR1 e.g., as set forth in GenBank accession nos. NM_001964.2 and NP_001955.1 (mRNA and protein, respectively).

Thus, according to one embodiment, the compounds of the present invention downregulate an activity or expression of EGR1 by about 10%, 20%, 30%, 40%, 50%, 60%, 70%, 80%, 90% or 100% as compared to the activity or expression of the EGR1 in a cell of the subject prior to the treatment (or in a corresponding sample of another subject having the same pathology and preferably matched with the same species e.g. human, age, weight, sex etc. as the subject in need thereof).

According to some of any of the embodiments described herein, the skin disease is associated with expression of SAMD9 and/or EGR1.

According to some of any of the embodiments described herein, the skin disease is treatable by downregulating an expression and/or activity of EGR1.

According to some of any of the embodiments described herein, the skin disease is treatable by inducing SAMD9 transcription activity and thereby downregulating an expression and/or activity of EGR1.

According to some of any of the embodiments described herein, the skin disease is associated with deficiency of SAMD9, for example, in a diseased skin area or tissue.

According to some of any of the embodiments described herein, the inflammation and/or autoimmune and/or hyperproliferative disease is associated with expression of SAMD9 and/or EGR1.

According to some of any of the embodiments described herein, the inflammation and/or autoimmune and/or hyperproliferative disease is treatable by downregulating an expression and/or activity of EGR1.

According to some of any of the embodiments described herein, the inflammation and/or autoimmune and/or hyperproliferative disease is treatable by inducing SAMD9 transcription activity and thereby downregulating an expression and/or activity of EGR1.

According to some of any of the embodiments described herein, the inflammation and/or autoimmune and/or hyperproliferative disease is associated with deficiency of SAMD9.

Downregulation of EGR1 can be determined and/or measured using methods well known in the art, including, for example, those described in the Examples section that follows.

Transcription activity of SAMD9 can be determined and/or measured using methods well known in the art, including, for example, those described in the Examples section that follows.

Pharmaceutical Compositions and Modes of Administration:

In any of the methods and uses described herein, the CPT compounds can be administered to a subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the CPT compounds as described herein which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to one embodiment, the CPT compounds are formulated for cutaneous e.g., topical administration (e.g., to a keratinous tissue, such as the skin, scalp), subcutaneous, dermal, or transdermal administration. For example, the pharmaceutical composition of some embodiments of the invention is formulated as a cream, lotion, spray, ointment, salve, gel, oil, wash, etc. for applying or spreading onto the surface of the body, i.e. skin, scalp, hair, nails and the like, preferably on the surface or in close proximity to the inflammation (e.g. psoriasis).

The pharmaceutical composition can be administered in a local manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

According to some of any of the embodiments described herein, in any of the methods and uses described herein, the CPT compound or the pharmaceutical composition comprising said administered to the skin, for example, to a diseases region of the skin, or to diseased skin cells.

According to some of any of the embodiments described herein, in any of the methods and uses described herein, the CPT compound or the pharmaceutical composition comprising said administered topically, for example, to a diseases region of the skin, or to diseased skin cells.

According to some of any of the embodiments described herein, the pharmaceutical composition is formulated for application to the skin, for example, to a diseases region of the skin, or to diseased skin cells.

According to some of any of the embodiments described herein, the pharmaceutical composition is formulated for topical application, or for topical application to the skin, for example, to a diseased region of the skin, or to diseased skin cells.

The phrases “formulated for topical application to skin” and “topical administration” are meant to define interchangeably terms that encompasses the formulation of the CPT compound into a dosage form that can be applied to skin of a subject and which result in the local presence of the compound in the skin. The phrase “local presence of the compound in skin” is meant to include topical administration of the active ingredient to skin with the presumption that systemic uptake of the active ingredient is limited or null. Thus, it is intended that less than 25% by weight, or less than 20% by weight, or less than 15% by weight, or less than 10%, 8%, 5% and 3% by weight, of the topically administered active ingredient enters the blood stream or is recovered in urine and faeces.

According to some of any of the embodiments described herein, the pharmaceutical composition for topical application, which is also referred to herein as a dermatological composition, is formulated such that the CPT compound contacts, or is locally present in, the epidermis and/or the upper dermis. According to some of these embodiments, the systemic uptake of the CPT compound is limited or null, as described herein.

According to some of any of the embodiments described herein, a pharmaceutical composition for application to the skin comprises a dermatologically acceptable carrier.

The term “dermatologically acceptable,” as used herein, means that the ingredients of the carrier are suitable for use in contact with human keratinous tissue, in particular diseased skin tissue or cells without undue toxicity, incompatibility, instability, allergic response, and the like.

For topical application or administration, the pharmaceutical composition can be formulated in any of a variety of forms utilized by the pharmaceutical industry for skin application including solutions, lotions, sprays, creams, ointments, salves, gels, oils, wash, etc., as described below.

The pharmaceutical compositions of the present invention may be formulated viscous enough to remain on the treated skin area, does not readily evaporate, and/or is not easily removed by rinsing with water, but rather is removable with the aid of soaps, cleansers and/or shampoos.

Methods for preparing compositions having such properties are well known to those skilled in the art, and are described in detail in Remington's Pharmaceutical Sciences, 1990 (supra); and Pharmaceutical Dosage Forms and Drug Delivery Systems, 6th ed., Williams & Wilkins (1995).

The topical compositions of the present embodiments, including but not limited to, lotions and creams, may comprise a dermatologically acceptable emollient. As used herein, “emollient” refers to a material useful for the prevention or relief of dryness, as well as for the protection of the skin. Wide varieties of suitable emollients are known and may be used herein. See, e.g., Sagarin, Cosmetics, Science and Technology, 2nd Edition, Vol. 1, pp. 3243 (1972), which contains numerous examples of materials suitable as an emollient and is fully incorporated herein by reference. Exemplary emollients include, but are not limited to, glycerin, hydrocarbon oils and waxes, such as mineral oil, petrolatum, and the like, vegetable and animal oils and fats, such as olive oil, palm oil, castor oil, corn oil, soybean oil, and the like, and lanolin and its derivatives, such as lanolin, lanolin oil, lanolin wax, lanolin alcohols, and the like.

The topically applied pharmaceutical composition of the present embodiments may also include additional components which are added, for example, in order to enrich the pharmaceutical compositions with fragrance and skin nutrition factors.

Such components are selected suitable for use on human keratinous tissue without inducing toxicity, incompatibility, instability, allergic response, and the like within the scope of sound medical judgment. In addition, such optional components are useful provided that they do not unacceptably alter the benefits of the active compounds of the present embodiments.

The pharmaceutical compositions of the present invention can be applied directly to the skin. Alternatively, it can be delivered via normal skin application by various transdermal drug delivery systems which are known in the art, such as transdermal patches that release the composition into the skin in a time released manner. Other drug delivery systems known in the arts include pressurized aerosol bottle, iontophoresis or sonophoresis. Iontophoresis is employed to increase skin permeability and facilitate transdermal delivery. U.S. Pat. Nos. 5,667,487 and 5,658,247 discloses an ionosonic apparatus suitable for the ultrasonic-iontophoretically mediated transport of therapeutic agents across the skin. Alternatively, or in addition, liposomes or micelles may also be employed as a delivery vehicle.

The pharmaceutical composition may be formulated as a unit dosage form. In such form, the preparation is subdivided into unit doses containing appropriate quantities of the active ingredients such as for a single administration. The unit dosage form can be a packaged preparation, the package containing discrete quantities of preparation, for example, an adhesive bandage, a non-adhesive bandage, a wipe, a baby wipe, a gauze, a pad and a sanitary pad.

Pharmaceutical compositions for topical application, which are also referred to herein throughout as dermatological compositions, may be provided in several designs such as in the form of an emulsion including a microemulsion and a liposome formulation, gel, solution, liniment, ointment, foam, spray, aerosol, microsponge, patch or powder.

In considering applying a dermatological composition to diseased skin and to achieve satisfactorily delivery of the CPT compound through the stratum corneum into the dermis, the composition may be adapted to such conditions. For example, diseased skin, such as skin affected by a dermatological disease as defined herein, may suffer from disrupted surface and with less intact stratum corneum. Therefore, dermatological compositions which are easy to apply on diseased skin, in particular onto greater parts of the skin, and which results in evenly spreading of the CPT compound throughout the diseased skin is desirable. Such compositions are thought to encompass emulsions, gels, solutions, sprays, foams and aerosols, preferable emulsions that are soft and easy to apply to diseased skin.

Dermatological compositions can be water-based. The content of a hydrophilic phase may be critical to the proper penetration profile, sufficient retention of the CPT compound in the diseased skin cells, if desired. A water-based dermatological composition can contain more than 40% of a hydrophilic phase by weight of the vehicle.

Dermatological compositions may further include a fatty component or oily component, which may constitute up to between 20 and 70% of the carrier.

If desired, the pH of the hydrophilic phase may be adjusted by adding acceptable acids or bases such as diethanolamine, citric acid, ascorbic acid, lactic acid, triethanolamine, sodium hydroxide, hydrochloric acid and sodium phosphate. The pH of the hydrophilic phase of the dermatological composition may be in the range between 3 and 8, but preferably in the range from pH 5 and 8, even more preferably about 6.5 and 7.8.

A dermatological composition according to some of the present embodiments may be in one of the following forms:

A water-in-oil emulsion: The CPT compound may be incorporated into an emulsion that includes a continuous phase of a hydrophobic phase and an aqueous phase that includes water and optionally one or more polar hydrophilic carrier(s) and salts. These emulsions may include oil-soluble or oil-swellable polymers as well as one or more emulsifier(s) that help to stabilize the emulsion.

An oil-in-water emulsion: The CPT compound may be emulsified into an emulsion comprising a discrete phase of a hydrophobic phase and a continuous aqueous phase that includes water and optionally one or more polar hydrophilic carrier(s) as well as salts, surfactants, emulsifiers, and other components. These emulsions may include water-soluble or water-swellable polymers as well as one or more emulsifier(s) that help to stabilize the emulsion.

A hydrophobic ointment: The CPT compound can be formulated into a hydrophobic base (e.g. by incorporating in petrolatum, thickened or gelled water, insoluble oils, triglycerides, transcutol and the like) and optionally with a minor amount of a water soluble phase.

Thickened Aqueous gels: CPT compound can be formulated into an aqueous phase which has been thickened to achieve a high viscosity. The thickening can be furnished by suitable natural, modified natural or synthetic polymers as described below. Alternatively, the thickened aqueous gels can be thickened using suitable polyethoxylated alkyl chain surfactants that effectively thicken the composition as well as other non-ionic, cationic, or anionic emulsifier systems. Preferably, non-ionic emulsifier systems are chosen since ionic emulsifiers tend to be sensitive to the salt content. Examples on non-ionic systems are Polawax, Cosmowax, and Crothix emulsifying systems.

Hydrophilic gels: The CPT compound can be formulated into a continuous phase that includes at least one water soluble hydrophilic component other than water. The formulations may contain water up to about between 70 and 99% by weight, such as between 80 and 95% by weight. Lower levels may be suitable in some compositions. Suitable hydrophilic components include glycols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof, and the like.

Foams: The CPT compound can be formulated as foam that typically include as the vehicle water, a lipophilic solvent, a surface-active agent, an emulsifier and a specific gelling agent. Such carriers, when placed in an aerosol container and combined with a liquefied gas propellant, create a non-translucent oil-in-water emulsion that is stable in its pre-dispensed state. Liquefied gas propellant is added to the carrier in an amount of about 3-18% by weight of the total composition. Upon release from the aerosol container, the carriers form breakable foam products, which are suitable for topical administration.

Exemplary constituents that that are usable or may be included in the dermatological compositions include, but are not limited to, the following:

Oily components, which are constituents of the hydrophobic phase of the various dermatological compositions forms and which may be made of one of the following dermatologically acceptable ingredients or a mixture of two or more thereof: almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and teaseed oil), mineral oils, fatty oils, liquid paraffin, mineral oil, isopropyl myristate, beewax, cottonseed oil, cetosteraryl alcohol, lanolin, white soft paraffin, yellow soft paraffin, canola oil, cetyl alcohol (cetanol), peanut oil, oleic acid, isopropyl palmitate, castor oil, stearyl alcohol, jojoba oil, stearic acid and silicone oils.
Fatty components, which are constituents of the hydrophobic phase of the various dermatological compositions forms and may be used in combination with or instead of the oil phase and typically includes one or more ingredients selected from beeswax, paraffin, petrolatum, triglycerides, cetyl palmitate, vegetable oils, sorbitan esters of fatty acids (Span), solid macrogols (polyethylene glycols), and condensation products between sorbitan esters of fatty acids and ethylene oxide, e.g. polyoxyethylene sorbitan monooleate (Tween). Typical fatty components may be selected from the group comprising petrolatum, paraffins, vegetable oils, animal fats, synthetic glycerides, waxes, lanolin, and liquid polyalkylsiloxanes. Typical fatty components are but not limited to solid macrogols (polyethylene glycols).

Aqueous phase, which constitutes the hydrophilic phase and which mainly comprise water, hydrophilic solvents, surfactants, emulsifier, preservatives, pH adjusters, flavors, colors and other hydrophilic ingredients.

Hydrophilic solvents which may be added to the aqueous phase, such as polar solvents in the form of water, propylene glycol, glycerol, sorbitol, ethanol, industrial methylated spirit, polyethylene glycols, propylene glycols, propylene carbonate, and triacetin.

Lipophilic solvents which may be added to the lipophilic phase, such as non-polar solvents in the form of isopropyl alcohol and medium chain triglycerides (MCT).

Emollients, such as fatty acid mono, di or tri glycerides, and fatty acid esters, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Emulsifiers (emulsifying agents), which may be added either to the aqueous phase or to the oil phase: Compositions of the present invention can include one or more emulsifiers to emulsify the composition. As used herein the term “emulsifier” means an amphiphilic molecule possessing both polar and non-polar regions which are covalently bound and capable of reducing the surface tension of water and for the interfacial tension between water and an immiscible liquid. The term is meant to include soaps, detergents, emulsifiers, surface active agents, and the like. The emulsifier can be cationic, anionic, non-ionic, or amphoteric. This includes a wide variety of conventional emulsifiers;

Non-ionic Emulsifiers. Exemplary non-ionic emulsifiers include, but are not limited to, Polyol esters including glycols (e.g. ethylene glycol, diethylene glycol, glycol stearate and propylene glycol monoesters of fatty acids (propylene glycol stearate, propylene glycol oleate or propylene glycol palmitostearate)) and glycerol esters (e.g. glyceryl stearate, glyceryl monooleate, glycerylmonolaurate, glyceryl ricinolate, glyceryl monocaprylate);

Sorbitan derivatives, that consists of esters of cyclic anhydrides of sorbitol with a fatty acid (C12-C18). Sorbitan derivatives are divided into two groups i) sorbitan esters of fatty acids (e.g. sorbitan monolaurate, sorbitan monooleate, sorbitan monostearate (SPAN 60™), sorbitan monopalmitate, sorbitan sesquioleate, sorbitan trioleate or sorbitan tristearate) and ii) polyoxyethylene sorbitan esters (e.g. polyoxyethylene sorbitan monostearate (TWEEN 60™), polyoxyethylene sorbitan tristearate (TWEEN 65™), polyoxyethlene sorbitan monooleate (TWEEN 80™);

Polyoxyethylene esters (also called macrogol esters) are mixtures of mono- or di-fatty acids esters (from C12 to C18) of polyoxyethylene glycol (PEG), e.g. stearate esters of PEG (PEG-40, PEG-50 and PEG-55), laurate, oleate, and myristate esters of PEG; Polyoxyethylene ethers are ethers of macrogol and fatty alcohols, such as ethers of the alcohols: stearyl (steareth emulsifiers), cetosteraryl (ceteareth emulsifiers) and oleyl (oleth emulsifiers);

Poloxamers that are polyoxyethylene-polyoxypropylene derivatives with polyoxyethylene groups (e.g. poloxamers-188);

Nonylphenyl ethers (nonoxinols) that are ethoxylated nonylphenols;

Propylene glycol Diacetate;

Polyvinyl alcohol;

Alkanolamides prepared from reaction of fatty acids with mono or diethanolamine; Fatty alcohols (e.g. cetyl alcohol and stearate alcohol); alkyl glucosides; alkyl polyglucosides; polyhydroxy fatty acid amides; sucrose esters; fatty acid alkanolamides; ethoxylated fatty acids; ethoxylated aliphatic acids; ethoxylated fatty alcohols (e.g., octyl phenoxy polyethoxyethanoal available under the trade name TRITON X-100 and nonyl phenoxy poly(ethyleneoxy)ethanol available under the trade name NONIDET P-40, both from Sigma, St. Louis, Mo.); ethoxylated and/or propoxylated aliphatic alcohols; ethoxylated glycerides; ethoxylated propoxylated block copolymers such as PLURONIC and TETRONIC surfactants available from BASF.

Cationic emulsifiers, including, but are not limited to: salts of primary, secondary, or tertiary fatty amines that optionally may be polyoxyalkylenated; quaternary ammonium salts, such as tetraalkylammonium, alkylamidoalkyltrialkylammonium, trialkylbenzylammonium, trialkylhydroxyalkylammonium, or alkylpyridinium halides (preferably chlorides or bromides) as well as other anionic counter-ions, such as but not limited to, alkyl sulfates, such as but not limited to, methosulfate and ethosulfate; imidazoline derivatives; amine oxides of a cationic nature (e.g., at an acidic pH). Examples of amineoxide emulsifiers include those which are lauryldimethylamine oxide, laurylamidopropyldimethylamine oxide, and cetyl amine oxide.

Anionic emulsifiers, including, but are not limited to, sarcosinates, glutamates, alkyl sulfates, sodium or potassium alkyleth sulfates, ammonium alkyleth sulfates, ammonium laureth-n-sulfates, laweth-n-sulfates, isethionates, glycerylether sulfonates, sulfosuccinates, alkylglyceryl ether sulfonates, alkyl phosphates, aralkyl phosphates, alkylphosphonates, and aralkylphosphonates. These anionic emulsifiers may have a metal or organic ammonium counterion.

Amphoteric emulsifiers, including, but not limited to, emulsifiers having tertiary amine groups, which may be protonated, as well as quaternary amine containing zwitterionic emulsifiers. Examples of such amphoteric emulsifiers include, but are not limited to: certain betaines such as cocobetaine and cocamidopropyl betaine; monoacetates such as sodium lauroamphoacetate; diacetates such as disodium lauroamphoacetate; amino- and alkylamino-propionates such as lauraminopropionic acid. Ammoniurn Sulfonate Amphoterics. This class of amphoteric emulsifiers refers to “sultaines” or “sulfobetaines”, such as cocamidopropyl-hydroxysultaine.

Exemplary emulsifiers are those that have an HLB (i.e., hydrophilic to lipophilic balance) of at least 4 and more preferably at least 6. Even more preferred emulsifiers are hydrophilic emulsifiers having an HLB in the range between 8 and 20, such as in the range between 10 and 20. Most preferred emulsifiers have an HLB of at least 12, such as at least 15. One or more emulsifiers may be used in the compositions of the present invention at a suitable level to produce the desired result. In a preferred embodiment, the one or more emulsifier are present in a total amount of at least 0.1 wt %, more preferably at least 0.5 wt %, and even more preferably at least 1.0 wt %, based on the total weight of the ready to use composition. In order to avoid irritation of a emulsifier in a preferred embodiment, the emulsifier is present in a total amount of no greater than 10 wt %, more preferably no greater than 5 wt %, even more preferably no greater than 3 wt %, and even more preferably no greater than 2 wt %, based on the total weight of the ready to use composition.

Polymeric thickeners which may be added to the hydrophilic phase; e.g. gums such as acacia, alginates, carageenan, chitosan, collagen, tragacanth and xantham; celluloses, such as sodium carboxymethyl-, hydroxymethyl-, hydroxypropyl- and hydroxypropylmethyl celluloses; acrylic acids, such as carbomers and polycarbophil; colloidal solids such as silica, clays and microcrystalline cellulose; hydrogels such as polyvinyl alcohol and polyvinylpyrrolidone; thermoreversible polymers such as poloxamers.

pH adjuster (buffering agents) which may be added to the hydrophilic phase, such as diethanolamine, lactic acid, monoethanolamine, triethanolamine, sodium hydroxide, sodium phosphate, citric acid, acetic acid, tartaric acid, hydrogen phosphoric acid, phosphate salts and diethylamine.

Permeation enhancers, which may be added either to the hydrophilic or lipophilic phase in order to increase the penetration of the CPT compound within stratum corneum.

Preservatives, such as antimicrobial agents like benzalkoniumchloride, benzyl alcohol, chlorhexidine, imidazolidinyl urea, phenol, potassium sorbate, benzoic acid, bronopol, chlorocresol, parabens esters, phenoxyethanol and sorbic acid.

Humectants, such as glycerol, glycerine, propylene glycol, sorbitol.

Chelating agents, such as citric acid and edetic acid.

Antioxidants, such as alfa-tocopherol, ascorbic acid, ascorbyl palmitate, butylated hydroxyanisole, butylated hydroxytoluene, sodium ascorbate, sodium metabisulphite

Suspending agents that may be selected from the group comprising celluloses and cellulose derivatives such as, e.g., carboxymethyl cellulose, hydroxyethylcellulose, hydroxypropylcellulose, hydroxypropylmethylcellulose, carrageenan, acacia gum, arabic gum, tragacanth, and mixtures thereof.

Gel-forming agents (thickeners), including gel bases and viscosity-increasing components such as, but not limited to, liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch, or cellulose derivatives such as, e.g., carboxymethylcellulose, hydroxyethylcellulose and other cellulose derivatives, water-swellable hydrocolloids, carrageenans, hyaluronates (e.g. hyaluronate gel optionally containing sodium chloride), and alginates including propylene glycol alginate. Still other examples are high molecular weight polysaccharide gum, such as xanthan gum.

In some embodiments, the pharmaceutical composition is prepared by uniformly and intimately bringing the active ingredient into association with a liquid carrier or a finely divided solid carrier or both, and placing it in appropriate packaging. In exemplary embodiments, the topical formulation described herein is placed in an appropriate container such as, for example, a squeeze-tube with a cap for dispensing ointments and creams, or a device for dispensing unit dosages of the drug (such as a bottle or dropper that dispenses a controlled pre-determined dosage of the active agent to a target area.

For topical administration, compound I may be formulated as a solution, gel, ointment, cream, suspension, etc. as are well-known in the art.

Exemplary formulations formulated for topical administration, include a therapeutically effective amount of a compound of Formula A or a pharmaceutically acceptable salt thereof, a topical base, an antioxidant, an emollient, and an emulsifier.

In exemplary embodiments, the therapeutically effective amount of the CPT compound may vary, and may range from 0.01% to 10% by weight, of the total weight of the formulation, including any intermediate values and subranges therebetween.

The topical base may comprise, for example, polyethylene glycol having a selected molecular weight, as a topical base. In some embodiments, the polyethylene glycol is of a molecular weight of from 3000 to 8000 Daltons.

In some embodiments, the formulation is an ointment, and may further include a water-miscible solvent, such as a polyalkylene glycol having an average molecular weight of from 200 Daltons to 600 Daltons. In some embodiments, the water-miscible solvent comprises PEG-400, and even more particularly PEG-400 substantially free of impurities. In certain embodiments, PEG-400 substantially free of impurities comprises less than 65 ppm formaldehyde, less than 10 ppm formaldehyde, or 1 ppm or less formaldehyde.

Topical formulations for use as described herein also can include a penetration enhancer, such as dimethyl isosorbide, propylene glycol, or combinations thereof; an emollient, such as water; a surfactant, such as sorbitan monostearate, a polyethylene glycol monostearate, D-α-tocopheryl polyethylene glycol 1000 succinate, a composition comprising glycol stearate/PEG32 stearate/PEG6 stearate, and combinations of surfactants; an antioxidant, such as butylated hydroxyanisole, butylated hydroxytoluene, ascorbic acid, a tocopherol, and combinations thereof, with particular embodiments comprising butylated hydroxytoluene as an antioxidant; and an optional colorant, such as 0.05% to 0.25% (w/w) caramel colorant.

In one embodiment, the formulation is a solution. In another embodiment, the formulation is a gel. In another embodiment, the formulation is a suspension. In yet another embodiment, the formulation is a cream or ointment. In one embodiment, the formulation is a liquid, for example, a homogeneous liquid or a suspension, sold in a bottle which dispenses the formulation as drops or a liquid film (for example, from an applicator tip that contacts a target area of the skin to dispense the liquid substantially only on a target area of the skin to be treated). In one embodiment, the formulation is a cream or ointment, sold in a tube which dispenses the formulation to a target area of the skin. In another embodiment, the CPT compound is provided in a viscous liquid (such as carboxylmethylcellulose, hydroxypropylmethycellulose, polyethylene glycol, glycerin, polyvinyl alcohol, or oil containing drops) for rubbing into the skin.

The formulations may have preservatives or be preservative-free (for example in a single-use container). One embodiment is any of the aforementioned formulations in a kit for topical or local administration.

Topical formulations optionally may comprise additional pharmaceutically acceptable ingredients such as diluents, stabilizers and/or adjuvants.

The topical formulations may include one or more excipients selected from solvents, topical bases, surfactants/emulsifiers, penetration enhancers, emollients, antioxidants, color additives, and any combination thereof.

Exemplary topical bases include, but are not limited to, hydrophobic vehicles such as hydrocarbons, liquid petrolatum (mineral oil, liquid paraffin, paraffin oil), white petrolatum (petroleum jelly, VASELINE®), yellow petrolatum (petroleum jelly), squalane (perhydrosqualene, spinacane), and silicones; silicones such as liquid polydimethylsiloxanes (dimethicone, silastic, medical grade silicone oil), alcohols such as lauryl alcohols (1-dodecanol, dodecyl alcohols), myristyl alcohols (tetradecanol, tetradecyl alcohols), cetyl alcohols (hexadecanol, ethal, palmityl alcohols), stearyl alcohols (stenol, cetosteryl alcohols), oleyl alcohols (ocenol); sterols such as sterol esters; lanolin such as hydrous wool fat, lanum; anhydrous lanolin (such as wool fat, anhydrous lanum, agnin); semi synthetic lanolins; carboxylic acids such as lauric acid, myristic acid, palmitic acid, stearic acid, oleic acid; esters and polyesters, such as cholesterol esters (stearate), ethylene glycol monoesters, propylene glycol monoesters, glyceryl monoesters, glyceryl monostearate, sorbitol monoesters, sorbitan monoesters, sorbitol diesters, sorbitan polyesters (spans, arlacels), glyceryl tristearate, lard, almond oil, corn oil, castor oil, cottonseed oil, olive oil, soybean oil, hydrogenated oils, sulfated oils, isopropyl myristate, isopropyl palmitate; and ethers and polyethers (polydisperse or monodisperse), such as polyethylene glycols or polypropylene glycols (pluronics).

Exemplary water-miscible solvents that may be used include polyols and polyglycols such as propylene glycol (1,2-propanediol), glycerin (glycerol), liquid polyethylene glycol, solid polyethylene glycol (hard macrogol, Carbowax®), glycol furol, 1,2-phenol-hexanetriol, sorbitol solution, esters and polyesters such as polyoxyethylene sorbitan monoesters (e.g., Tween® 60) and polyoxy ethylene sorbitan polyesters (e.g., Tween® 20), ethers and polyethers such as polyethylene glycol monocetyl ether (cetomacrogol 1000) and polyethylene-polypropylene glycols (pluronics).

Exemplary surfactants include, but are not limited to a sterol or sterol ester, for example cholesterol (cholesterin), lanolin (hydrous wool fat, lanum), anhydrous lanolin (wool fat, anhydrous lanum, agnin), or semi synthetic lanolin; carboxylic acids such as Na+, K+, ethanolamine salts of lauric acid, myristic acid, palmitic acid, stearic acid, oleic acid, or an ether or polyether such as polyethylene-polypropylene glycols (pluronics). If an oil-in-water (o/w) emulsifier is desired, the following examples may be used: esters and polyesters such as polyoxyethylene, sorbitan monoesters (Span™ 20, Span™40, Span™ 80), polyoxy ethylene esters (stearate-polyethylene glycol monoesters, Myrj®45, Myrj®59), polyoxy ethylene sorbitan polyesters (tweens); ethers and polyethers such as polyethylene glycol monocetyl ether (cetomacrogol 1000) or polyethylene-polypropylene glycols (pluronics), and others such as sodium lauryl sulfate, borax (sodium borate), ethanolamine, or triethanolamine. Nonionic surfactants, like Surfactant 190 (dimethicone copolyol), Polysorbate 20 (Tween®20), Polysorbate 40 (Tween®40), Polysorbate 60 (Tween.® 60), Polysorbate 80 (Tween® 80), lauramide DEA, cocamide DEA, and cocamide MEA, amphoteric surfactants like oleyl betaine and cocamidopropyl betaine (Velvetex® BK-35), and cationic surfactants, like Phospholipid PTC (Cocamidopropyl phosphatidyl PG-dimonium chloride), can be used. Appropriate combinations or mixtures of such surfactants may also be used.

Exemplary penetration enhancers include, but are not limited to, alcohol, alkyl methyl sulfoxide, pyrrolidone, laurocapram, dimethyl formamide, tetrahydrofurfuryl alcohol, an amphiphile, or other miscellaneous enhancers such as clofibric acid amide, hexamethylene lauramide, dimethyl isosorbide, propylene glycol, proteolytic enzymes, terpenes or sesquiterpenes.

Exemplary moisturizers include, but are not limited to, lactic acid and other hydroxy acids and their salts, glycerin, propylene glycol, butylene glycol, sodium PCA, Carbowax®200, Carbowax®400, and Carbowax®800. Suitable emollients for use in the formulations include, but are not limited to, water, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl® 424 (myristyl myristate), octyl dodecanol, dimethicone (Dow Corning 200-100 cps), phenyl trimethicone (Dow Corning 556), Dow Corning 1401 (cyclomethicone and dimethiconol), and cyclomethicone (Dow Corning 344), and Miglyol®840 (manufactured by Huls; propylene glycol dicaprylate/dicaprate), including any combination thereof.

The pharmaceutical composition may also include preservatives, antimicrobials, and/or antioxidants, such as benzalkonium chloride, benzoic acid, benzyl alcohol, bronopol, chlorhexidine, chlorocresol, imidazolidinyl urea, paraben esters, phenol, phenoxyethanol, potassium sorbate, or sorbic acid; antioxidants such as .alpha.-tocopherol, ascorbic acid, ascorbyl palmitate, butylated hydroxyanisole, butylated hydroxytoluene, sodium ascorbate, sodium metabisulfite; chelating agents such as citric acid or edetic acid; buffers such as citric acid and salts, phosphoric acid and salts, H₃PO₄/NaH₂PO₄, glycine, acetic acid, triethanolamine, or boric acid; humectants such as glycerin (glycerol), propylene glycol (E1520), glyceryl triacetate (E1518), sorbitol (E420), xylitol and malitol (E965), polydextrose (E1200), quillaia (E999), lactic acid, urea or lithium chloride; and/or a sequestering antioxidant such as citric acid and it salts ethylenediaminetetraacetic acid (Versene®, EDTA).

The pharmaceutical composition further may include dyes/colorants and/or fragrances. Suitable fragrances and colors, such as caramel, FD&C Red No. 40 and FD&C Yellow No. 5, may be used in the formulations. Other examples of fragrances and colors suitable for use in topical products are known in the art.

Other suitable additional and adjunct ingredients which may be included in the formulations include, but are not limited to, absorbents (e.g., hydrogels), astringents (e.g., witch hazel, alcohol, and herbal extracts such as chamomile extract), binders (e.g., starch, cellulose ethers, microcrystalline cellulose, calcium hydrogen phosphate, calcium phosphate dibasic, and calcium sulfate dihydrate), other excipients (e.g., polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose), buffering agents (e.g., monobasic or dibasic potassium phosphate, monobasic or dibasic sodium phosphate, magnesium hydroxide), chelating agents (e.g., EDTA (ethylenediaminetetraacetic acid, tetrasodium salt)), film-forming agents (e.g., chitosan, hydroxypropylmethylcellulose, polyvinyl alcohol), conditioning agents (e.g., petrolatum, glycerin, propylene glycol), opacifying agents (e.g., titanium dioxide), pH adjusters (e.g., citric acid and sodium hydroxide), and protectants. Examples of each of these ingredients, as well as examples of other suitable ingredients in topical product formulations, may be found in publications by The Cosmetic, Toiletry, and Fragrance Association (CTFA). See, e.g., CTFA Cosmetic Ingredient Handbook, 2nd edition, eds. John A. Wenninger and G. N. McEwen, Jr. (CTFA, 1992).

In an exemplary ointment formulation, the composition is based on petrolatum. The ointment does not contain sufficient water to separate into a second phase at room temperature. A water-soluble ointment may be formulated with polyethylene glycol. The ointment can be in a convenient container such as a tube or jars.

In an exemplary embodiment, the formulation is a cream in which the compounds are dissolved or suspended in water removable or emollient bases. The creams may be either water-in-oil or oil-in-water compositions. Immiscible compounds may be combined by mechanical agitation or heat using wet gum, dry gum, bottle, and beaker methods. In some embodiments, the cream is an oil-in-water emulsion or aqueous microcrystalline dispersion of long chain fatty acids or alcohols that are water washable and more cosmetically and aesthetically acceptable.

In exemplary embodiments, the formulation is in a form of a paste, which can be considered an ointment into which a high percentage of insoluble solids have been added, for example as much as 50% by weight. Ingredients such as starch, zinc oxide, calcium carbonate, and talc are used as the solid phase. In exemplary embodiment, the formulation is in a form of a gel, jelly or lotion. Gels are semisolid systems consisting of dispersions of small or large molecules in an aqueous liquid vehicle rendering jelly-like through the addition of gelling agent. Among the gelling agents used are synthetic macromolecules, such as carbomer 934, and cellulose derivatives, such as carboxymethylcellulose or hydroxypropylmethyl-cellulose. The gels may be either single-phase gels in which the macromolecules are uniformly distributed throughout a liquid with no apparent boundaries between the dispersed macromolecules and the liquid, or double-phase gels in which the gel mass consists of floccules of small distinct particles, often referred to as a magmas. A jelly contains a water-soluble base typically prepared from natural gums such as tragacanth, pectin, alginate, or boroglycerin, or from synthetic derivatives of a natural substance such as methylcellulose or carboxymethylcellulose. A lotion is a clear solution containing 25-50% alcohol, which optionally contains an antiseptic, or mollient. Other optional ingredients that may be added to the lotion are an extract of witchhazel, menthol, glycerin, boric acid, alum, or potassium oxyquinoline.

In exemplary embodiments, the topical formulation may include a topical base, a water-miscible solvent, a penetration enhancer, an emollient, a surfactant, an antioxidant, a colorant, or any combination thereof, in addition to a CPT compound as described herein.

In exemplary embodiments, the formulation comprises from 0.01% to 10% by weight of a CPT compound, from 15% to 25% (w/w) topical base, from 40% to 50% (w/w) water-miscible solvent, from 10% to 20% (w/w) penetration enhancer, from 5% to 15% (w/w) emollient, from 3% to 7% (w/w) surfactant, from 0.5% to 1.5% (w/w) antioxidant, and optionally from 0.05% to 0.25% (w/w) colorant.

In exemplary embodiments, the pharmaceutical formulation includes from 0.1% to 10% (w/w) CPT compound, from 15% to 40% (w/w) topical base, from 25% to 50% (w/w) water-miscible solvent, from 10% to 20% (w/w) penetration enhancer, from 3% to 15% (w/w) emollient, from 3% to 9% (w/w) surfactant, from 0.5% to 1.5% (w/w) antioxidant, and optionally from 0.05% to 0.25% (w/w) colorant.

In exemplary embodiments, the topical base is selected from polyethers such as polyalkylene glycols. Suitable polyalkylene glycols include polyethylene glycols (e.g., PEG with an average molecular weight ranging from 3000-8000 Daltons). The polyethylene glycol may be polydisperse or monodisperse. The water-miscible solvent may be a polyether such as a liquid polyalkylene glycol (e.g., PEG with an average molecular weight ranging from 200-600 Daltons). In some embodiments, the penetration enhancer is dimethyl isosorbide (DMI), propylene glycol, or a combination thereof. In some embodiments, the emollient is water. Suitable surfactants include Tefose® 63 (glycol stearate, PEG32 stearate, PEG6 stearate), Span® (sorbitan monostearate), Myrj® (polyethylene glycol monostearate), or TPGS (D-α-tocopheryl polyethylene glycol 1000 succinate). In some embodiments, the antioxidant is BHT (butylated hydroxytoluene). Caramel may be added as a colorant.

Any other combinations of ingredients and/or of an amount thereof are contemplated.

In some embodiments, the CPT compound is included in a pharmaceutical composition for topical application in which the CPT compound and/or other active ingredients in the formulation is/are encapsulated in metal oxide particles such as particles prepared by the sol-gel methodology. Non-limiting examples of such compositions are described in U.S. Patent Application having publication Nos. 2018/0339176, 2018/0207451, 2018/0147165, 2018/0117369, 2014/0147396, 2013/0018023, 2012/0321685, 2012/0295790, 2012/0202695, 2012/0015014, 2011/0262506, 2011/0177951, 2011/0150954, 2010/0255107, 2010/0203121, 2010/0047357, 2010/0016443, 2009/0081262, and in U.S. Pat. Nos. 8,110,284, 8,039,020, 7,758,888, 6,303,149, 10,420,743, 9,868,103, 9,687,465, 9,561,485, 8,449,918, 8,425,940, the contents of which are incorporated by reference as if fully set forth herein.

In some embodiments, the CPT compound in formulated in a pharmaceutical composition for topical application and/or carrier and/or device such as described in U.S. Patent Application Publication Nos. 2013/0189191, 2013/0183251, 2013/0183250, 2013/0184242, 2013/0189196, 2013/0189193, 2013/0164225, 2013/0189195, 2013/0195769, 2012/0087872, 2014/0066524, 2010/0310476, 2009/0130029, 2011/0281827, 2005/0075407, 2013/0053353, 2005/0074414, 2010/0040561, 2014/0050673, 2004/0265240, 2009/0317338, 2010/0284938, 2014/0227199, 2006/0140984, 2005/0069566, 2014/0241998, 2008/0152596, 2008/0069779, 2006/0088561, 2006/0275221, 2010/0111879, 2010/0137198, 2012/0213709, 2015/0017103, 2006/0193789, 2008/0206159, 2018/0235984, 2009/0041680, 2014/0271494, 2014/0121188, 2012/0213710, 2018/0193469, 2014/0086848, 2006/0275218, 2007/0020213, 2009/0180970, 2010/0266510, 2014/0248219, 2013/0011342, 2019/0022000, 2010/0221195, 2013/0064777, 2006/0285912, 2007/0069046, 2019/0029958, 2008/0031907, 2011/0008266, 2019/0022001, 2013/0161351, 2008/0044444, 2019/0247310, 2011/0212033, 2006/0233721, 2005/0031547, 2012/0237453, 2008/0253973, 2014/0193502, 2009/0068118, 2014/0182585, 2019/0282501, 2008/0138296, 2008/0317679, and 2011/0097279; and U.S. Pat. Nos. 10,213,384, 10,363,216, 9,884,017, 9,161,916, 10,398,641, 8,343,945, 8,618,081, 10,265,404, 9,668,972, 9,795,564, 8,518,376, 8,435,498, 8,741,265, 8,722,021, 8,486,375, 9,492,412, 7,700,076, 8,795,693, 8,840,869, 6,967,023, 6,911,211, 9,101,662, 8,512,718, 10,092,588, 10,350,166, 9,572,775, 7,682,623, 9,167,813, 6,994,863, 9,713,643, 7,645,803, 8,636,982, 9,662,298, 8,114,385, 10,238,746, 10,137,200, 8,945,516, 10,369,102, 8,362,091, 10,029,013, 9,072,667, 8,119,109, 9,682,021, 8,795,635, 9,622,947, 9,636,405, 7,704,518, 8,709,385, 10,117,812, 10,322,085, 9,265,725, 9,463,919, 8,900,554, 9,549,898, 8,978,936, 8,900,553, 8,617,100, 9,320,705, 9,539,208, and 8,486,374, the contents of each is incorporated by reference as if fully set forth herein.

In some embodiments, the CPT compound in formulated in a pharmaceutical composition for topical application and/or carrier and/or device such as described in U.S. Patent Application Publication Nos. 2011/0008267, 2007/0254953; and/or U.S. Pat. Nos. 4,767,032, 9,918,960, 9,724,324, 5,804,215, 9,789,057, 7,445,796, 10,278,917, 8,920,821, and 9,452,137, the contents of which are incorporated by reference as if fully set forth herein.

The quantity of active compound in a unit dose of preparation may be varied or adjusted according to the particular application.

Pharmaceutical compositions suitable for use in context of some embodiments of the invention include compositions wherein the active ingredients are contained in an amount effective to achieve the intended purpose. For example, a therapeutically effective amount means an amount of active ingredients (a CPT compound) effective for preventing, treating or reducing the inflammatory response (e.g. anti-inflammatory effect) in a skin of a subject. A “therapeutically effective amount” of a CPT compound may be determined in a routine manner by any method known to one of skill in the art (e.g., blood test, ultrasound, X-ray, CT scan, MRI, visual inspection, and other means used for determining a skin condition in a subject).

Thus, determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide ample levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to some embodiments, the therapeutically effective amount of a CPT compound that is administered topically per day (daily) is lower than a therapeutically effective amount per day (daily) of CPT compounds that are known as usable for treating cancer or other proliferative diseases or disorder when administered systemically (e.g., by injection). In some of these embodiments, the therapeutically effective amount per day is lower by at least 20% compared with an amount per day of a CPT compound that is used for treating cancer or other proliferative diseases or disorder when administered systemically (e.g., by injection).

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In any of the methods and uses described herein, the CPT compound can be used or formulated with an additional active agent that is usable in treating the skin disease, inflammation and/or the hyperproliferative disease, as described herein.

In some embodiments, the additional active agent is other than a terpenoid compound as described in KR Patent Application No. 2011/010609.

In some embodiments, the additional active agent is other than a galectin-3 inhibitor as described in U.S. Patent Application Publication No. 2004/0223971.

In some embodiments, the additional active agent is other than a compound as described in CN Patent Application No. 109553608.

In some embodiments, the additional active agent is other than a non-covalent DNS binding agent as described in U.S. Patent Application Publication No. 2015/0056192.

In some embodiments, the additional active agent is other than an FGF antagonist as described in U.S. Patent Application Publication No. 2004/0010001.

In some embodiments, the additional active agent is other than an inositol compound as described in U.S. Patent Application Publication No. 2009/0214474.

In some embodiments, the additional active agent is other than amonafide and derivatives thereof as described in WO 2014/179528.

In some of any of the embodiments described herein, the CPT compound of Formula A is not linked covalently to an additional active agent or drug.

### Definitions

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein throughout, the phrase “linking moiety” describes a group (a substituent) that is attached to another moiety in the compound via two or more atoms thereof. In order to differentiate a linking group from a substituent that is attached to another moiety in the compound via one atom thereof, the latter will be referred to herein and throughout as an “end group”.

As used herein, the term “amine” describes both a —NR′R″ end group and a —NR′— linking group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, oxo, oxime, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine, oxo, oxime, thiohydrazine, hydrazide, thiohydrazide, silyl or hydrazine, or any of the substituents as described herein.

The term “amine” is used herein to describe a —NR′R″ group in cases where the amine is an end group, as defined hereinunder, and is used herein to describe a —NR′— group in cases where the amine is a linking group.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine oxo, oxime, thiohydrazine, hydrazide, thiohydrazide, silyl or hydrazine, or any of the substituents as described herein.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

The phrase “alkene” or “alkenyl”, as used herein, are an alkyl, as defined herein, which contains one or more double bonds.

The phrase “alkyne” or “alkynyl”, as used herein, are an alkyl, as defined herein, which contains one or more triple bonds.

Whenever an alkyl group is described herein as a substituent, it can be replaced by alkene or alkyne or allyl, as described herein.

The term “allyl” refers to a —C—CR′CR″R′″ end group or a —C—CR′═CR″— linking group, with R′, R″ and R′″ are as defined herein.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine, oxo, oxime, thiohydrazine, hydrazide, thiohydrazide, silyl or hydrazine, or any of the substituents as described herein. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine, oxo, oxime, thiohydrazine, hydrazide, thiohydrazide, silyl or hydrazine, or any of the substituents as described herein. The heteroalicyclic group can be an end group, as this phrase is defined hereinabove, where it is attached to a single adjacent atom, or a linking group, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine, oxo, oxime, thiohydrazine, hydrazide, thiohydrazide, silyl or hydrazine, or any of the substituents as described herein. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine, amine-oxide, oxo, oxime, thiohydrazine, hydrazide, thiohydrazide, silyl or hydrazine, or any of the substituents as described herein. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “amine-oxide” describes a —N(OR′)(R″) or a —N(OR′)— group, where R′ and R″ are as defined herein. This term refers to a —N(OR′)(R″) group in cases where the amine-oxide is an end group, as this phrase is defined hereinabove, and to a —N(OR′)— group in cases where the amine-oxime is an end group, as this phrase is defined hereinabove.

The term “halide” and “halo” describes fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein.

The term “N-sulfonamide” describes an R'S(═O)₂—NR″— end group or a —S(═O)₂—NR′— linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “phosphonate” describes a —P(═O)(OR′)(OR″) end group or a —P(═O)(OR′)(O)— linking group, as these phrases are defined hereinabove, with R′ and R″ as defined herein.

The term “thiophosphonate” describes a —P(═S)(OR′) (OR″) end group or a —P(═S)(OR′)(O)— linking group, as these phrases are defined hereinabove, with R′ and R″ as defined herein.

The term “phosphinyl” describes a —PR′R″ end group or a —PR′— linking group, as these phrases are defined hereinabove, with R′ and R″ as defined hereinabove.

The term “phosphine oxide” describes a —P(═O)(R′)(R″) end group or a —P(═O)(R′)— linking group, as these phrases are defined hereinabove, with R′ and R″ as defined herein.

The term “phosphine sulfide” describes a —P(═S)(R′)(R″) end group or a —P(═S)(R′)— linking group, as these phrases are defined hereinabove, with R′ and R″ as defined herein.

The term “phosphite” describes an —O—PR′(═O)(OR″) end group or an —O—PH(═O)(O)— linking group, as these phrases are defined hereinabove, with R′ and R″ as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an —N═C═O group.

The term “nitro” describes an —NO₂ group.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “peroxo” describes an —O—OR′ end group or an —O—O— linking group, as these phrases are defined hereinabove, with R′ as defined hereinabove.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

The term “N-carbamate” describes an R″OC(═O)—NR′— end group or a —OC(═O)—NR′— linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′— linking group, as these phrases are defined hereinabove, with R′ and R″ as defined herein.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

The term “guanyl” describes a R′R″NC(═N)— end group or a —R′NC(═N)— linking group, as these phrases are defined hereinabove, where R′ and R″ are as defined herein.

The term “guanidine” describes a —R′NC(═N)—NR″R′″ end group or a —R′NC(═N)—NR″— linking group, as these phrases are defined hereinabove, where R′, R″ and R′″ are as defined herein.

The term “hydrazine” describes a —NR′—NR″R′″ end group or a —NR′—NR″— linking group, as these phrases are defined hereinabove, with R′, R″, and R′″ as defined herein.

The term “silyl” describes a —SiR′R″R′″ end group or a —SiR′R″— linking group, as these phrases are defined hereinabove, whereby each of R′, R″ and R′″ are as defined herein.

The term “siloxy” describes a —Si(OR′)R″R′″ end group or a —Si(OR′)R″— linking group, as these phrases are defined hereinabove, whereby each of R′, R″ and R′″ are as defined herein.

The term “silaza” describes a —Si(NR′R″)R′″ end group or a —Si(NR′R″)— linking group, as these phrases are defined hereinabove, whereby each of R′, R″ and R′″ is as defined herein.

The term “silicate” describes a —O—Si(OR′)(OR″)(OR′″) end group or a —O—Si(OR′)(OR″)— linking group, as these phrases are defined hereinabove, with R′, R″ and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

As used herein, the term “epoxide” describes an end group or a linking group, as these phrases are defined hereinabove, where R′, R″ and R′″ are as defined herein.

As used herein, the term “methyleneamine” describes an —NR′—CH₂—CH═CR″R′″ end group or a —NR′—CH₂—CH═CR″— linking group, as these phrases are defined hereinabove, where R′, R″ and R′″ are as defined herein.

As used herein, the term “alkylene glycol” describes a —O—[(CR′R″)_{z}—O]_{y}R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is an oligo(alkylene glycol).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND EXPERIMENTAL PROCEDURES

Materials:

Camptothecin (CPT) was obtained from the National Cancer Institute (NCI) in the National Institutes of Health (NIH).

Irinotecan (CPT-11) was obtained from Pfizer.

These commercially available drugs were selected as exemplary molecules of Formula 1. An aqueous solution containing 1% DMSO, 5% ethanol and 20% lipofuscin was used as a vehicle, unless otherwise indicated. Whenever the vehicle is indicated as DMSO or as DMSO in lipofuscin it is meant the above-described aqueous solution.

Cell Cultures:

HeLa cells were cultured in DMEM 4.5 g/l glucose medium containing 10% FCS, 1% L-glutamine and 1% penicillin and streptomycin (Biological Industries) and grown at 37° C. and 5% CO₂.

TERC-transformed fibroblasts were transformed with a pBABE-H2AGFP construct containing the human telomerase gene were cultured High Glucose DMEM, 10% FCS, L-glutamine and Pen-Strep (all cell culture regents from Biological Industries, Israel). Medium was changed every 2-3 days. Cells were used at passage 2-3.

Primary fibroblasts were isolated from human adult skin and were grown in High Glucose DMEM, 10% Fetal Bovine Serum, L-glutamine and Pen-Strep. Medium was changed every 2-3 days. Cells were used at passage 2-3.

Of note, when cells were incubated with CPT and CPT-11, they were grown in the appropriate medium with 0.1% FCS only.

Mice Having Imiquimod-Induced Psoriasiform Dermatitis:

Balb/c mice (Harlan Laboratories Ltd, Jerusalem, Israel), 9 weeks of age, were maintained in a pathogen-free animal facility. Animal care and research protocols had been approved by the institutional committee for animal use. Imiquimod 5% (Perrigo, Israel) was topically applied to the mice on their upper backs.

Chimeric Mice:

Chimeric mice carrying human psoriatic skin were generated using SCID mice, 2-3 months of age. Animals were maintained in a pathogen-free animal facility. A psoriasis-like phenotype was induced in normal human skin grafted onto the mice by intradermal injection of natural killer/T cells derived from psoriatic patients as previously described [Gilhar A et al. J Invest Dermatol (2011) 131: 118-124].

Light microscopy and immunohistochemistry:

Formaldehyde-fixed 5-μm paraffin-embedded sections from skin biopsies were deparaffinized and treated with 3% H₂O₂ in methanol for 15 minutes at room temperature, warmed in a microwave oven in citrate buffer in a pressure cooker for 25 minutes, and stained with a monoclonal anti-Ki67 antibody (Thermo Scientific) for 1 hour at room temperature. Following 3 washings (10 minutes each) with phosphate-buffered saline (PBS), the antibodies were imaged using the ABC technique (Zymed Laboratories, South San Francisco, Calif.) and the slides were counterstained with hematoxylin.

Epidermal thickness was defined as the distance between the granular layer and the basement membrane and was measured at 10 randomly selected locations for each biopsy using NIS-Elements BR 3.2 software (Nikon, NY USA). An image was taken and was manually measured in the software by selecting an upper and lower border, as defined above. The distance was measured in microns.

Quantitative Reverse Transcription PCR (qRT-PCR):

RNA was extracted from cell cultures using an RNA extraction kit (Roche Diagnostics, Mahheim, Germany). cDNA was synthesized from 500 ng of total RNA using the Verso cDNA kit (Thermo Fisher Scientific, Waltham, Mass., USA) or qScript kit (Quanta Biosciences, Gaithersburg, Md., USA). cDNA PCR amplification was carried out using the Fast SYBR Green Master Mix in a StepOnePlus™ Real-Time PCR System (Applied Biosystems, Foster City, Calif., USA) with gene-specific intron-crossing oligonucleotide pairs listed in Table 1 (below). For quantification, standard curves were obtained using serially diluted cDNA amplified in the same qRT-PCR run. The melting temperature (Tm) of the amplified products was measured to confirm the specificity of the reaction conditions. Cycling conditions were as follows: 95° C. for 10 minutes, 95° C. for 10 seconds, 62° C. for 15 seconds, and 72° C. for 25 seconds for a total of 40 cycles. Each sample was analyzed in triplicate. mRNA expression level for target gene was normalized to GAPDH. The results are based the amount of target, normalized to an endogenous reference (IACTB) and relative to a calibrator, as calculated by 2-ΔΔCT. All samples were run in triplicate. The expression levels of target genes were expressed as mRNA relative units.

TABLE 1
Oligonucleotide sequences.

| Gene Name | Sequence | SEQ ID NO. |
|---|---|---|
| SAMD9 F | 5′-CGAGCAAGGTCCTTCCATAGTG-3′ | 1 |
| SAMD9 R | 5′-CCGATGACCTCACAGCTCAAG-3′ | 2 |
| EGR1 F | 5′-GGGCAGTCGAGTGGTTTGG-3′ | 3 |
| EGR1 R | 5′-TTGCCGACAGGATGCAGAAGGA-3′ | 4 |

### Example 1

### Effect of CPT and CPT-11 on SAMD9 Expression in Human Cell Cultures

The potential ability of CPT and CPT-11 to induce SAMD9 expression in cell cultures was tested.

SAMD9 expression was measured via qRT-PCR, in triplicates.

HeLa Cells, TERC-transformed fibroblasts and primary fibroblasts were cultured in 12-well plates and treated with 5 μM of CPT in DMSO for 24 hours in duplicates. The obtained data is presented in FIG. 1A and shows significant induction of SAMD9 expression in all the tested cell lines.

FIG. 1B presents the SAMD9 expression in primary fibroblasts in the presence of DMSO only or 1 μM, 2 μM, 5 μM and 10 μM CPT. As can be seen, treatment with CPT up-regulate SAMD9 expression in a dose dependent manner.

The effect of CPT on SAMD9 expression and EGR1 repression in Hela cells was also tested. Cells were cultured in duplicates in 12-well plates in the presence of DMSO or 10 μM of CPT for 24, 48 and 72 hours. SAMD9 and EGR1 expression were measured via qRT-PCR, all samples were run in triplicates.

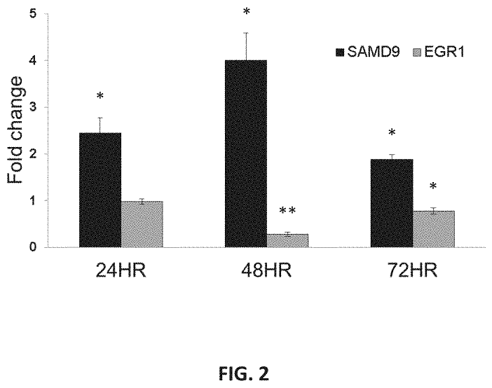

The obtained data is presented in FIG. 2. As can be seen, CPT also significantly down-regulated EGR1 expression in primary fibroblast cells, albeit at different time points.

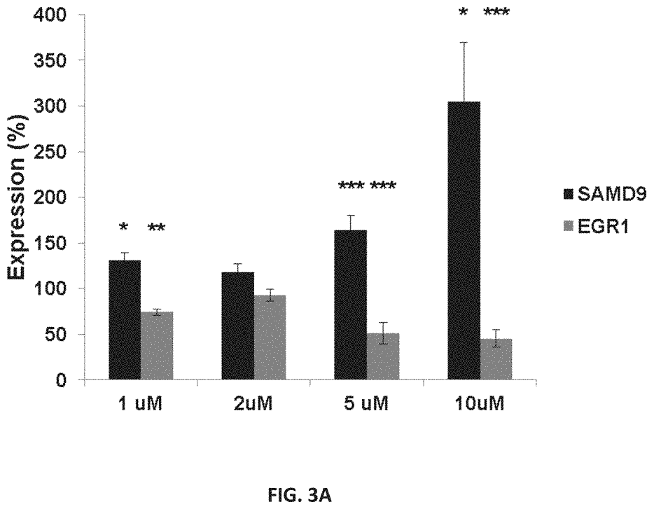

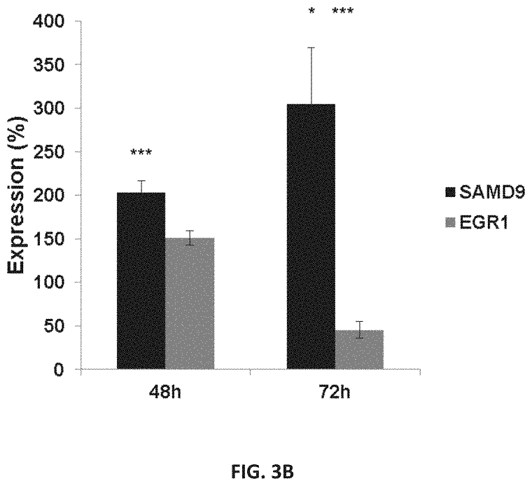

The effect of CPT-11 on SAMD9 expression and EGR1 repression in Hela cells was tested similarly. Cells were cultured in duplicates in 12-well plates in the presence of DMSO or 1 μM, 2 μM, 5 μM and 10 μM CPT-11 for 48 hours, or with 10 μM of CPT-11, or DMSO for 48 and 72 hours. SAMD9 and EGR1 expression were measured via qRT-PCR, all samples were run in triplicates. The obtained data is presented in FIGS. 3A and 3B, and show substantial induction of SAMD9 expression and substantial down-regulation of EGR1.

### Example 2

### Effect of CPT on Imiquimod-Induced Psoriasiform Dermatitis in Mice

The ability of the tested compounds to down-regulate the expression of EGR1, and thereby reduce skin inflammation was tested in a murine model in which psoriasiform dermatitis was induced using imiquimod [as previously described in Tortola, L. et al. (2012) J Clin Invest 122, 3965-76].

Balb/c mice were treated five times a week topically with imiquimod and were injected i.p. with either DMSO (as the vehicle), or 7.5 mg/Kg CPT.

At day 6, biopsies were obtained from the treated skin and stained for hematoxylin and eosin (H&E) as well as for Ki67, as a surrogate marker for cellular proliferation.

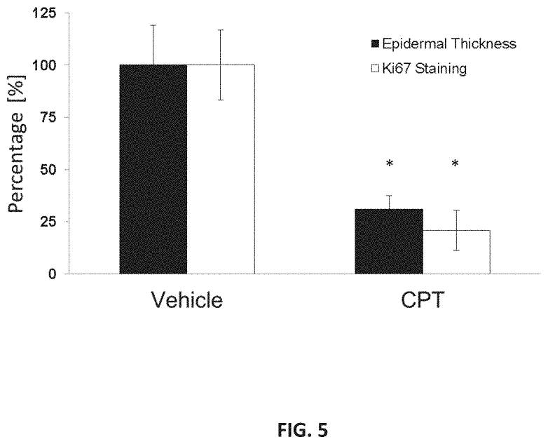

The obtained data is presented in FIGS. 4A-D and 5. As can be seen, treatment with CPT resulted in a decrease in epidermal thickness and in the index of proliferation (FIG. 5). These findings suggest a possible effect of CPT on the epidermal elements during psoriasis development.

### Example 3

### Effect of CPT and CPT-11 on Chimeric Mice Carrying Human Psoriatic Skin

To assess the potential therapeutic role of the compounds of some embodiments of the invention, the two compounds were administered systemically (i.p) to chimeric mice carrying human psoriatic skin.

In short, SCID mice, 2-3 months of age, were maintained in a pathogen-free animal facility. A psoriasis-like phenotype was induced in normal human skin grafted onto the mice by intradermal injection of natural killer/T cells derived from psoriatic patients as previously described [Gilhar A et al. J Invest Dermatol (2011) 131: 118-124].

Two weeks after natural killer/T-cell injection (6 weeks after human skin grafting), mice were injected i.p. five times a week. Five groups of mice were treated as follows: one group of mice was injected five times a week with the vehicle (10% DMSO in lipofuscin was used as a vehicle control); a second group of mice was injected five times a week CPT (3 mg/kg); a third group of mice was injected three times a week CPT-11 (50 mg/kg); a fourth group of mice was injected three times a week CPT-11 (30 mg/kg); and a fifth group of mice, was treated with dexamethasone (DEX) cream applied 5 times a week on the graft, as positive control (DEX was expected to attenuate inflammation in this model). Each group included six mice, and the treatment was performed for a total of 10 days. The grafts were harvested from the five groups of mice, paraffin-embedded, stained for hematoxylin and eosin (H&E), analyzed and scored for the average improvement of the clinical and histological psoriasiform phenotype.

As shown in FIG. 6, treatment with both compounds (CPT and CPT-11) resulted in significant clinical and histological attenuation of the psoriasiform phenotype. As shown in FIGS. 7A-H, 8 and 9, biopsies obtained from mice skin after 10 days and stained for hematoxylin and eosin as well as for Ki67, as a surrogate marker for cellular proliferation, showed a significant decrease in epidermal thickness akeratinocyte proliferation and on the inflammation index (as was determined by examination of the inflammatory infiltrate in the histological slides) in chimeric mice carrying human psoriatic skin.

These findings show the effect of CPT and CPT-11 on both epidermal and immune elements during psoriasis development and the significant anti-inflammatory effect of these compounds.

### Example 4

### RNA Sequence Analysis

HeLa cells were treated with 10 μM CPT or DMSO for 48 hours (three independent experiments). Total RNA was extracted and sent to RNA-sequencing.

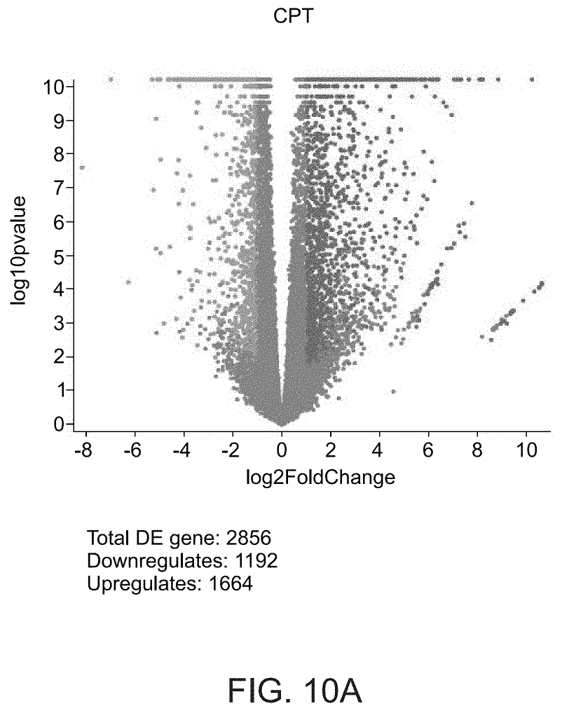

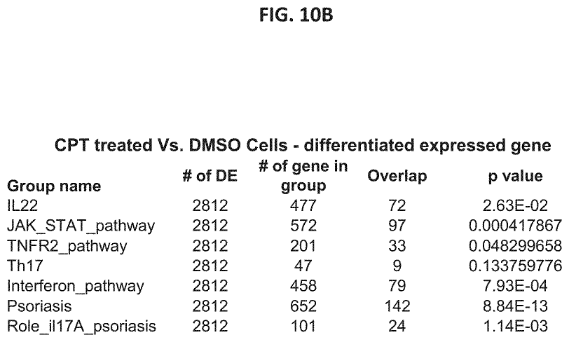

The obtained data is presented in FIGS. 10A-B. FIG. 10A presents a volcano plot that show the number of statistically significant differential gene (DE) as response to CPT treatment. The treatment caused significant impact on global gene expression with hundreds of DE genes (1192 gene were downregulated, while 1664 genes were upregulated). In addition, calculation of p-value for enrichment for genes belongs to a known psoriasis related pathogenic pathways (taken from IPA database) using hypergeometric distribution (using a background of 24190 genes). The table in FIG. 10B summarizes the number of genes in each group, the number of differentially expressed genes, the overlap, and the adjusted p-value (after correcting for multiple testing). This analysis showed significant turnover in expression of many genes belongs to psoriasis related pathways.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating an inflammatory skin disease in a subject in need thereof, the method comprising administered to the subject a therapeutically effective amount of a pharmaceutical composition comprising a compound represented by Formula A: 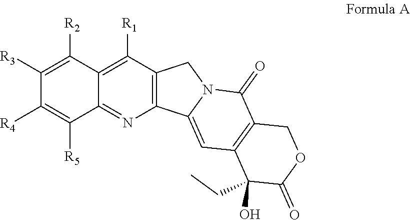
or a pharmaceutically acceptable salt thereof, or a carboxylate form thereof,
wherein:
R₁-R₅ are each independently selected from hydrogen, alkyl, alkenyl, allyl, cycloalkyl, halo, trihaloalkyl, amino, alkoxy, thioalkoxy, hydroxyl, thiol, nitro, cyano, aryl, heteroaryl, silyl, oxime, carboxylate, thiocarboxylate, carbamate, thiocarbamate, or, alternatively or in addition, two of R₁-R₅ form together a cyclic ring, said cyclic ring being selected from aryl, heteroaryl, cycloalkyl or heteroalicyclic, each being independently substituted or unsubstituted,
wherein the pharmaceutical composition is formulated for topical application, and comprises water and an oily or fatty component.

2. The method of claim 1, selected from camptothecin, irinotecan, topotecan, and SN-38.

3. The method of claim 1, wherein said compound is capable of inducing SAMD9 expression and/or of downregulating EGR1.

4. The method of claim 1, wherein the skin disease is a chronic inflammatory skin disease.

5. The method of claim 1, wherein the skin disease is an acute inflammatory skin disease.

6. The method of claim 1, wherein the skin disease is selected from the group consisting of an atopic dermatitis, a contact dermatitis, a dermatitis herpetiformis, a generalized exfoliative dermatitis, a seborrheic dermatitis, a psoriasis, a drug rash, an erythema multiforme, an erythema nodosum, a granuloma annulare, a poison ivy, a poison oak, a toxic epidermal necrolysis, an acne and a rosacea.

7. The method of claim 1, wherein the skin disease is selected from atopic dermatitis and psoriasis.

8. The method of claim 1, wherein the pharmaceutical composition is configured or formulated such that said compound is locally present in the epidermis and/or upper dermis.

9. The method of claim 1, wherein the pharmaceutical composition is configured or formulated such that a presence of said compound is the physiological system of the subject is minimized or null.

10. The method of claim 1, wherein the oily or fatty component is selected from beeswax, paraffin, petrolatum, a triglyceride, a vegetable oil, a sorbitan ester of a fatty acid, polyoxyethylene sorbitan monooleate (Tween), and an animal fat.

11. The method of claim 1, wherein an amount of said compound ranges from 0.01% to 10% by weight, of the total weight of the pharmaceutical composition.

12. The method of claim 1, wherein said carrier further comprises at least one of a water-miscible solvent, an emollient, a surfactant and an antioxidant.

13. The method of claim 1, wherein said carrier further comprises at least one of glycol stearate, PEG32 stearate, PEG6 stearate, sorbitan monostearate, polyethylene glycol monostearate, D-α-tocopheryl polyethylene glycol 1000 succinate, butylated hydroxytoluene, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, dimethicone, phenyl trimethicone, cyclomethicone, dimethiconol, cyclomethicone, propylene glycol dicaprylate/dicapratem cholesterol, polyoxyethylene, a sorbitan monoester, a polyoxy ethylene ester, a polyoxy ethylene sorbitan polyester, a polyethylene glycol monocetyl ether, a polyethylene-polypropylene glycol, sodium lauryl sulfate, borax (sodium borate), ethanolamine, triethanolamine, dimethicone copolyol, lauramide DEA, cocamide DEA, cocamide MEA, oleyl betaine, cocamidopropyl betaine, polyols, polyglycols such as propylene glycol (1,2-propanediol), glycerin (glycerol), liquid polyethylene glycol, solid polyethylene glycol, glycol furol, 1,2-phenol-hexanetriol, sorbitol solution.


SEQUENCE LISTING (4 sequences)

SEQ ID NO: 1 - DNA, 22 bp, synthetic construct
  misc_feature 1..22: note = Single strand DNA oligonucleotide
  source 1..22: mol_type = other DNA
cgagcaaggtccttccatagtg

SEQ ID NO: 2 - DNA, 21 bp, synthetic construct
  misc_feature 1..21: note = Single strand DNA oligonucleotide
  source 1..21: mol_type = other DNA
ccgatgacctcacagctcaag

SEQ ID NO: 3 - DNA, 19 bp, synthetic construct
  misc_feature 1..19: note = Single strand DNA oligonucleotide
  source 1..19: mol_type = other DNA
gggcagtcgagtggtttgg

SEQ ID NO: 4 - DNA, 22 bp, synthetic construct
  misc_feature 1..22: note = Single strand DNA oligonucleotide
  source 1..22: mol_type = other DNA
ttgccgacaggatgcagaagga